(19) United States
(12) Patent Application Publication
(10) Pub. No.: US 2014/0323993 A1
(43) Pub. Date: Oct. 30, 2014

(54) APPLICATOR

(71) Applicants: Alun Wilcox, Surrey (GB); Peter Sprada, Sydney (AU); Alexandra Louisa Faraday Crook, Sydney (AU); Steven William McLellan, Billericay (GB); Graham Keith Lacy, London (GB); Julian Francis Ralph Swan, London (GB)

(72) Inventors: Alun Wilcox, Surrey (GB); Peter Sprada, Sydney (AU); Alexandra Louisa Faraday Crook, Sydney (AU); Steven William McLellan, Billericay (GB); Graham Keith Lacy, London (GB); Julian Francis Ralph Swan, London (GB)

(73) Assignee: LEO PHARMA A/S, Ballerup (DK)

(21) Appl. No.: 14/007,349

(22) PCT Filed: Mar. 8, 2012
(86) PCT No.: PCT/GB12/50518

§ 371 Date: Jul. 3, 2014

Related U.S. Application Data
(60) Provisional application No. 61/467,610, filed on Mar. 25, 2011.

(51) Int. Cl. A61M 35/00

(52) U.S. Cl. CPC A61M 35/003
USPC 604/290; 604/311

(57) ABSTRACT

An applicator for topically applying a semi-solid medicament comprises an applicator head having an applicator face for spreading medicament across the skin; a sealable aperture on the applicator face; and a drive mechanism for forcing medicament out through the aperture. The applicator head includes a cartridge port at the rear of the applicator face for receiving a dispensing end of a cartridge charged with medicament. The drive mechanism comprises a dial knob rotatable around a longitudinal axis of the applicator for advancing a piston along a drive member towards the applicator head to force medicament from the cartridge and thereby dispense a predetermined dose of medicament onto the applicator face.

The present invention relates to an applicator for applying medicated preparations to the skin, especially for example to the skin of a person suffering from psoriasis. More particularly, the invention relates to an applicator for use with replacement medicament cartridges and to a cartridge for use with the applicator.

Psoriasis occurs when the usual cycle of replacing skin cells is accelerated. While psoriasis presents itself as a skin condition, in recent years it has been established that its origins lie in the immune system. Normally skin cells take about 21-28 days to replace themselves, but in psoriasis sufferers the skin cells can be replaced as rapidly as every 2-6 days. This acceleration results in an accumulation of skin cells on the skin surface, often referred to as psoriasis plaque.

Patches of psoriasis, or plaques, can occur more or less anywhere on the body, but most often occur on the elbows, knees, lower back and scalp. Typically, it appears as patches of raised, red skin covered in silvery white scales. The scales are the accumulation of skin cells that are waiting to be shed and the red patches occur as a result of an increase in the number of blood vessels necessary to support the raised volume of cell production.

Frequently, plaques are itchy and uncomfortable, or worse painful or sore and a range of treatments are available to alleviate the condition. Treatment options include topical therapies, phototherapy, systemic medication and biological injections. Treatment is selected according to the severity of the condition, but in most cases topical therapies are preferred, at least at the outset as they carry lower risks and are more convenient for the patient.

For many psoriasis sufferers, topical therapies, that is preparations applied directly to the skin, are sufficient to keep the condition under control. Conventional preparations include those based on one or more of vitamin D, coal tar, dithranol, vitamin A and steroids and are supplied as semi-solid formulations.

Usually, the formulations are prescribed by medical personnel together with a dosage regime defining the amount of medicament to be applied and the frequency of application. Medications are generally supplied in tubes from which an amount is squeezed out for application to the skin area being treated. Often, the amount to be applied is defined in terms of finger-tip units (FTUs), one finger-tip unit being the distance from the tip of an adult finger to the first crease of that finger, and the number of FTUs to be applied depends on the area of application, so the dose is determined according to the skin area being treated.

A number of psoriasis sufferers interviewed on behalf of the Applicant have indicated that reliable dosing is a significant problem. An FTU is an inaccurate measure since it will vary from person to person. If only a small area of skin is affected, a sub-FTU may be required which again is difficult to measure. Obviously, if insufficient medicament is applied, the affected skin will not be soothed or heal as well as it otherwise might under the correct dosage. On the other hand, application of medicament over and above the prescribed dose can have more serious consequences. For example, overdosing of topical treatments can cause severe irritation and burning. Overdosing of formulations such as steroid creams can result in absorption through the skin and unwanted side effects, such as those that may be experienced when systemic doses of steroids are taken.

One common side effect of regular steroid use is thinning or weakening of the skin. Even if steroid cream is applied to a psoriasis area in the correct FTU dosage, there will often be weakening of the skin at the user's fingertips from using them to rub in the cream. Wearing of gloves to apply steroid medication to avoid this problem is recommended but users find this cumbersome, messy and impractical.

Thus the Applicant has identified a need for a medicament applicator for applying semi-solid preparations, such as psoriasis ointment, to the skin which overcomes or at least mitigates one or more of the aforementioned problems.

The term medicaments or preparations used herein is intended to embrace medicated creams, gels, ointments and the like that can flow or be extruded and spread for topical application. The terms are used interchangeably throughout, thus references to ointments or the like should be construed as including other forms of semi-solid preparation.

From a first aspect, the invention resides in an applicator for topically applying a semi-solid medicament, the applicator comprising:
an applicator head having an applicator face for spreading medicament across the skin;
a sealable aperture on the applicator face; and
a drive mechanism for forcing medicament out through the aperture;
   wherein the applicator head includes a cartridge port at the rear of the applicator face for receiving a dispensing end of a cartridge charged with medicament, and wherein the drive mechanism comprises a dial knob rotatable around a longitudinal axis of the applicator for advancing a piston along a drive member towards the applicator head to force medicament from the cartridge and thereby dispense a predetermined dose of medicament onto the applicator face.

As will be appreciated, once the applicator has been primed, each forwards revolution of the dial knob will result in the piston advancing up the drive member by a predetermined distance to dispense a repeatable volume of medicament. The pitch of the thread on the drive rod may be selected so that, for example, a single revolution of the dial knob dispenses a typical unit dose of medicament. For example, each revolution may dispense 0.5 g of medicament, equivalent to what is normally regarded as a FTU. Of course, where smaller doses are required the dial knob may need to be rotated by an amount less than a complete revolution, such as a quarter or half-turn. In any event, each complete forwards revolution or fraction of a revolution will result in a known volume of medicament being dispensed.

By means of the present invention, a user is able to dispense a metered amount of medicament by manually rotating the dial knob by a single revolution or a multiple or fraction thereof, the medicament being dispensed through the aperture onto the applicator face from where it can be applied and spread over the skin to be treated. In doing so, there is no need for contact between the medicament and the user's fingers. Thus, for application of psoriasis ointment or the like, the user is not only reassured that the correct amount of ointment is being applied but also that the skin on his/her fingertips will not suffer through repeated contact with the ointment.

The applicator face is preferably smooth to enable all of the dispensed medicament to be transferred to the skin being treated and to provide a comfortable surface for rubbing against the skin. Moreover, a smooth surface may be readily wiped or cleaned after use to maintain hygiene. The applicator face may be substantially flat or may be gently convex to facilitate a slight rolling action of the applicator head during application of the medicament, and the outer perimeter is preferably rounded to ensure comfort against the skin during application.

Since skin affected by psoriasis is frequently inflamed and sore, the applicator face may be comprised of a metal. In this way, the applicator head can provide a cooling sensation to the skin at the same time as the medicament is applied. The applicator face may be made of a metal, such as aluminium, stainless steel, silver or the like, or an alloy, or of other materials, such as a natural or synthetic polymeric material, including a polymeric material having a metallic coating or incorporating a metal compound. In this regard, silver compounds may be particularly advantageous in view of inherent antimicrobial and healing properties thereof. Further materials that may be used for the applicator face include glass, natural and synthetic fibres, silicon, silicone, carbon and ceramics.

To make application of medicament using the applicator of the invention more comfortable and/or to make it easier for a user to apply medicament to areas of the body that are not readily accessible, it is preferred that the applicator face is inclined with respect to the longitudinal axis of the applicator.

Advantageously, the diameter of the applicator head flares out towards the applicator face, for example so that the applicator face has a diameter greater than the diameter of the applicator head in the region of the cartridge port thereby to create a shoulder portion behind the applicator face for shielding a user's fingers from medicament on the applicator face and from medicament applied to the skin when the applicator is in use. The shoulder portion is also helpful in achieving the desired degree of pressure, from the fingertips, when massaging the face against the skin.

To avoid contamination of the applicator face when the applicator is not in use, it is preferable to provide a cover therefor. Thus, the applicator according to the invention may further comprise a removable cover for fitting over the applicator face and retention on the applicator head. The removable cover may be retained on the applicator head by means of a friction fit, or may comprise a formation complementary to a formation on the applicator head to provide a positive engagement, for example an internal projection around the rim of the cover for engaging with an external groove around the applicator head or a part thereof. The removable cover may be flexible or resilient to allow the cover to be distorted or stretched over the applicator head during fitting.

A tab may be provided extending from the cover to assist a user in fitting and removing the cover, for example, application of downwards pressure on the tab can help drive a projection or rib on the underside of the cover, typically extending from the tab, into a groove or recess on the applicator head whereas application of pressure in an upwards direction helps disengage the projection or rib from the recess.

The applicator head may be supplied for use in combination with the cover and further comprise a foil seal adhered across the sealable aperture in the applicator face and attached to an internal surface of the cover, whereby upon removing the cover for the first time, the foil seal is peeled away from the aperture ready for use.

Preferably the sealable aperture on the applicator face is sealed by means of a valve, such as a one-way valve, thereby to avoid dispensed medicament from being forced back into the applicator when pressure is applied to the skin from the applicator face during application. A valve also serves to prevent other potential contaminants from entering the device through the aperture. Often, medicament is applied following bathing or showering, so the inclusion of a valve will prevent water from seeping in via the aperture if the applicator is accidentally dropped.

The valve for sealing the aperture in the applicator face may be incorporated with the applicator face itself, for example during or after manufacture thereof, or with the applicator head but not integrally with the applicator face. Alternatively, the valve may be integral with the cartridge for use with the applicator. In the latter case, when the cartridge is docked with the applicator head via the cartridge port, the valve on the cartridge may be forced up into the aperture to form a seal therewith. The valve may therefore also serve a dual purpose as a cartridge seal.

The aperture may be sealed by an elastomeric valve, such as valve manufactured from rubber or synthetic elastomer, for example a slit valve or a duckbill valve. Such elastomeric valves allow medicament to be dispensed in a controlled manner, opening to permit delivery of medicament when a force is applied to the medicament but closing as soon as the force is withdrawn, namely when movement of the drive mechanism is halted. An elastomeric valve may be readily incorporated with the applicator face, such as by moulding on the applicator head in a two-shot moulding process. The latter process enables the production of a seamless, easily cleanable joint between the applicator face and valve.

The aperture in the applicator face may alternatively be sealed by means of a pin valve, or a mushroom/poppet type valve. Such valves permit the size of the outlet through which the ointment is dispensed to be maximised, effectively the outlet is the same size as the aperture itself.

In one arrangement, the applicator face of the applicator is moveable from a first, rest position in which a pin or the like seals the aperture in the applicator face to a second, dispensing position in which the applicator face is raised in relation to the pin thereby to open the aperture and permit medicament to be dispensed. Advantageously, the applicator face may be moved from the first position to the second position under the pressure of the medicament when the dial knob is activated. This arrangement is advantageous in terms of permitting the medicament to be dispensed at a faster rate, and minimising or substantially removing any internal back pressure. As such, any tendency for ointment to ooze from the aperture once a dose has been dispensed is substantially eliminated. Moreover, as the applicator face is raised above the pin etc. the face itself remains safe and comfortable for use against a user's skin. With such an arrangement, once a dose of medicament has been dispensed on to the applicator face, the applicator face is returned to its first position, with the aperture once again sealed by the pin or the like, by the force exerted against the face by the skin of a user during topical application of the medicament.

Movement of the applicator face in relation to the pin or like seal may be achieved in a number of ways. For example, the applicator face may be moveable on the applicator head between first and second positions, or the applicator head itself may be moveable between first and second positions.

When the pin or the like is provided on the cartridge, at its dispensing end, the applicator head may comprise a moveable unit, wherein upon activation of the drive mechanism, the head is moved from the first, rest position to the second, raised position. In this way, when the cartridge is fitted to the cartridge port, the pin on the cartridge will seal the aperture when the applicator head is in its first position and the applicator head will be raised above the pin to open the aperture when the dial knob is rotated and pressure exerted on the medicament in the cartridge.

As will be appreciated, in such an arrangement, the cartridge port may also rise with the applicator head, thus the cartridge port is adapted to retain the cartridge irrespective of whether the applicator head is in the first or second position. For example, the applicator head may comprise an engagement mechanism for securing the head to a cartridge housing wherein the mechanism permits limited movement of the applicator head away from the housing. In this way, the applicator head may be raised to the extent necessary to open the aperture without becoming detached from the cartridge housing. In other words, the applicator head acts as a floating head with respect to the housing.

In another arrangement, a pin valve may be provided on the applicator head adjacent the cartridge port, but separate from the applicator face, and the applicator face comprises a moveable unit, moveable from a first, rest position in which the pin seals the aperture in the applicator face to a second, raised position above the pin. In this arrangement, when the cartridge is fitted to the cartridge port and the dial knob is rotated, the medicament under pressure in the cartridge exerts a force behind the applicator face thus raising the applicator face up above the pin and hence opening the aperture to allow medicament to be dispensed. In this arrangement, the cartridge may remain stationary in the cartridge port once inserted, and the applicator face acts as a floating fascia on the applicator head.

In a preferred arrangement, the valve comprises a pin valve that is retractable with respect to the applicator face. Thus in a first, raised position, the pin seals the aperture in the applicator face and in a second, retracted position the aperture is open to permit medicament to be dispensed through the aperture upon rotation of the dial knob. The aperture may be provided in a resilient insert that is fitted flush with the applicator face to help seal around the pin and close the aperture when the pin is in the raised position. Thus the insert is urged against the pin due to its resilience helping create an effective seal for avoiding ingress of dirt, etc.

In order to move the pin valve between raised and lowered positions, the applicator preferably further comprises a valve riser member operable on the pin valve to open or close the aperture. Preferably the pin riser member is interposed between the cartridge port and pin valve, the riser member, port and valve being axially aligned with each other and with the aperture in the applicator face. The pin valve may be provided with a projection that projects into the cartridge port for puncturing a frangible seal on the cartridge when fitting, for example the projection may terminate in a sharp tip, such as a spike, or have a serrated edge or the like.

The applicator preferably further comprises a switch mechanism for switching the valve between open and closed positions. For example, in relation to an applicator provided with a pin valve and valve riser member, the switch mechanism may comprise an external slider, suitable for fingertip operation, having an arm or the like captured in an elongate slot on the applicator for moving the valve riser member within the applicator head. The valve riser member elevates the valve with respect to the aperture upon operation of the slider in one direction or lowers the valve upon operation in the opposite direction.

The slider is preferably elongate such that it covers the elongate slot irrespective of whether it is in a “valve open” or “valve closed” position. Further, the slider may include an outwardly directed projection, that is on its external face, to assist a user when moving the slider between valve open and valve closed positions.

The elongate slot preferably extends substantially transverse to the longitudinal axis of the applicator; in this way the slider and hence the valve riser member are subject to a rotational movement with respect to the applicator. The valve riser member raises the valve to seal the aperture when the slider arm is moved along the slot to a first position, and lowers the valve to open the aperture when the slider arm is moved in a second, opposite direction. The valve riser member may, for example, have an inclined upwardly directed surface on which the valve is supported—rotation of the riser member in one direction results in the valve rising along the inclined surface and opposite rotation results in the valve being lowered along the inclined surface. The valve is preferably fully open when the slider is slid to a first, extreme position with respect to the slot and fully closed at the opposite extreme position.

In use, the slider is moved so that the valve opens the aperture preferably just prior to rotating the dial knob to dispense a dose of medicament, and is reversed so that the valve closes the aperture just after the medicament has been dispensed onto the applicator surface but prior to applying the medicament to the skin. In this way, foreign matter and dispensed medicament are substantially prevented from entering the aperture during application of the medicament thereby ensuring that the next dose dispensed from the applicator will be free of any contamination.

The applicator desirably includes indicia to inform a user as to whether the applicator, in particular the slider, is in a “valve open” position or a “valve closed” position. Such indicia may be aligned with the position of a projection on the slider when the slider is in each of its valve open and valve closed positions.

The cartridge port preferably comprises an open-ended tubular portion extending from behind the applicator face and into which the dispensing end of a cartridge containing medicament may be inserted. Most preferably, the cartridge port serves to align the cartridge outlet at the dispensing end thereof with the aperture in the applicator face and, if a pin valve or the like is provided, the cartridge port may also serve as a valve seat, preferably with a valve riser member therebetween. The tubular portion of the cartridge port may terminate in an inwardly directed flange for co-operating with a feature at or adjacent to the dispensing end of the cartridge to prevent the cartridge from being inserted too far into the applicator. For example, the dispensing end of the cartridge may comprise a neck having an external threaded portion adjacent the cartridge body for securing a threaded cartridge cap. The cap serves to keep the cartridge sealed prior to it being required for use in the applicator and is removed when the cartridge is fitted to the applicator. Thus the inwardly directed flange of the cartridge port may abut the (leading) end of the threaded portion of the cartridge neck when the cartridge has been inserted to the desired extent and prevent further insertion beyond this position.

The applicator head may include an upwardly inclined (i.e. directed towards the applicator face) internal shoulder against which a cartridge having a complementary downwardly directed shoulder adjacent its dispensing end may rest when fully inserted. The inclined shoulder may depend and flare out from the base of the tubular portion of the cartridge port. Such an inclined shoulder may also help guide the cartridge into position.

When fully inserted, the outlet at the dispensing end of the cartridge is preferably aligned with the aperture in the applicator face. If necessary, the applicator head may be provided with a flow passage, such as a bore, tube or the like, extending from the cartridge port to the aperture in the applicator face thereby to provide a continuous fluid flow path for medicament from the outlet at the dispensing end of the cartridge to the aperture. In a preferred arrangement, however, the cartridge outlet lies beyond the cartridge port when the cartridge is fully inserted, terminating proximate the valve that seals or opens the aperture on the applicator face.

The applicator preferably further comprises a retaining mechanism for securing the cartridge against the cartridge port in a fully inserted position. The cartridge retaining mechanism is most conveniently provided at or adjacent the cartridge port, for example, in the form of an internal screw thread on the cartridge port, such as on the aforementioned tubular portion, for co-operating with an external screw thread on the cartridge.

More preferably, the cartridge retaining mechanism comprises a pair of opposing L-slots for receiving opposing lugs on the cartridge, in the manner of a bayonet mounting. Preferably the slots are provided on the cartridge port. Thus the cartridge is fitted by initially pushing it into the cartridge port and twisting until the lugs “drop” into the slot openings, then the cartridge is twisted (rotated) further, such as by a quarter turn (90°), to secure the cartridge in place.

The applicator may further comprise a housing, or applicator body, for the cartridge which extends from the applicator head and encloses the cartridge, or a substantial part thereof, when the cartridge is in its fully inserted position. In one example, the applicator head and housing are provided with complementary fittings, such as an external screw thread on the applicator head and an internal screw thread an the upper, open end of the housing, or vice versa. In another example, the applicator head and housing are provided with complementary bayonet fixings, such as opposing L-shaped slots on the applicator head, typically on the tubular portion, and opposing pins on the housing.

In yet another option, the applicator head and housing may simply “click” together, with one of the applicator head or housing having a depressible projection, such as a button, and the other having an aperture into which the projection/button extends when fitted together. Separation of the applicator head from the housing may be achieved by depressing the projection/button to release the connection.

In a further, preferred option, the applicator housing extends from and is integral with applicator head. For example, the head and housing may comprise a single molding. Whilst the applicator face may also be integral with the applicator head, it preferably comprises a separate molding that is subsequently welded or otherwise joined to the applicator head to form a part thereof.

It is of course desirable that the dispensing mechanism of the applicator be easy-to-operate. This is especially important for patients suffering from psoriatic arthritis which typically affects the joints of the fingers making it difficult for the patient to operate devices that have controls that are fiddly or tricky to use. Thus dialing up a dose of medicament simply by rotating a dial knob on the applicator provides an easy means of dispensing the medicament. The dial knob is preferably provided with a textured or featured external surface to create a non-slip grip for the user. For example, the dial knob may have a plurality of raised grip features, preferably one of which is enlarged for helping a user relate to one full turn of the dial knob.

The dial knob may be provided on the cartridge itself at its non-dispensing end, or where the applicator comprises a housing for the cartridge, on the end of the housing adjacent to the non-dispensing end of the cartridge, effectively acting as an end cap on the cartridge or housing.

More preferably, the applicator comprises an applicator body and a dial assembly including the dial knob and a dial body, wherein the dial knob is rotatably mounted on the dial body. In order to insert and replace the cartridge, the dial body is preferably releasably attachable to the applicator body, such as by screw fitting, by a bayonet-type fixing, or other known interlocking mechanisms.

The applicator body preferably has a length such that when a cartridge is fully inserted, the non-dispensing end of the cartridge projects beyond the end of the applicator body, allowing the user to retain a grip on the cartridge throughout insertion and when subsequently removing the cartridge, for example when depleted and replaced. In addition, the projecting portion of the cartridge may carry information, such as one or more of the identity of the medicament, the manufacturer, its strength and expiry date, etc. Thus a user can be assured of the contents of the cartridge without having to detach the cartridge from the cartridge port.

The dial assembly and applicator body may include complementary indicators, such as an arrow on the dial body and unlocked and locked symbols on the applicator body for guiding a user when fitting the dial assembly to the applicator body. Thus, in the case of bayonet fixing, when the dial assembly is fitted on the applicator body, such as would occur after insertion of a cartridge, the arrow on the dial body is first aligned with the unlocked symbol and the dial body pushed in towards the applicator body and then twisted, for example a quarter turn, to lock the bodies together. In the locked condition, the arrow on the dial body will be aligned with the lock symbol on the applicator body.

The piston and drive member upon which the drive mechanism of the applicator acts may be provided with the cartridge, with a housing therefor, or with the dial knob, for example as part of a dial assembly comprising the dial knob. Accordingly, the drive member, typically a threaded drive rod, may be integral with, or attached to, the dial knob, so that the drive member is rotated with the dial knob. Such an arrangement is suitable for example for use with a cartridge of a type that does not have the drive member extending within it, such as a cartridge in the form of a flexible bag, as will be described more fully hereinafter.

An alternative arrangement, such as for use with a flexible bag-type cartridge discharged by means of an external piston, may comprise a threaded tube extending from the dial knob and rotatable therewith. The piston is in operable engagement with the threaded tube and in sealing engagement with an applicator body or bag housing and is driven along the threaded tube towards the applicator head as the dial knob is rotated, thereby compressing the medicament within the bag and from which medicament is forced out through the aperture in the applicator head.

In a preferred arrangement however, both the drive member, in the form of a threaded drive rod, and the piston are provided on the cartridge, one end of the drive rod projecting out from behind the piston contained within cartridge, the projecting end being operable upon by the dial knob to rotate the drive rod and advance the piston within the cartridge. In such an arrangement, the dial knob is preferably adapted to capture the projecting end of the drive rod or vice versa such that rotation of the dial knob effects rotation of the drive rod.

It is desirable for the drive rod to be coupled with the dial knob, for example after fitting the cartridge in the cartridge port of the applicator, without actuating the drive mechanism. Accordingly, the dial knob and drive rod are preferably brought into cooperation initially by linear coupling. For example, the dial knob may comprise a shaft or spindle for insertion into a corresponding bore in the end of the drive rod when the spindle and bore are axially aligned. Alternatively, the dial knob may have a bore for receiving the end of the axially aligned drive rod. The coupling spindle or rod and the bore preferably have complementary profiles such that after linear insertion of one into the other, the dial knob and drive rod rotate together upon operation of the dial knob. Such complementary profiles may simply involve a friction fit, but more preferably comprise non-circular cross-sections, such as polygonal, typically square or hexagonal, etc., or overlapping radial projections, or the end of the drive rod may be splined for meshing with longitudinal grooves in the bore of the dial knob.

The dial knob advantageously comprises a substantially hollow element having an outer dial ring and an inner bore in the form of a hollow spindle for receiving the end of the drive member. Most preferably, the bore of the dial knob has longitudinal grooves for engaging with a splined end of the drive rod.

In order to prevent accidental dispensing of medicament from the applicator, for example, when there is no valve switch, a dial knob deactivation mechanism may be provided which maintains the dial knob in an inoperable, rest position and returns the dial knob to the rest position after a dose of medicament has been dispensed. Such a mechanism may, for example, comprise a resilient member for urging the dial knob into the inoperable, rest position. Thus, in order for the dial knob to be actuated, a user must overcome the resilient force to move the dial knob to a second, operable position. Once the medicament has been dispensed, the user releases the dial knob whereupon the resilient force urges the knob back to its inoperable, rest position.

The deactivation mechanism may comprise a spring or other resilient element urging the dial knob into a rest position in which it is prevented from rotating, such as by means of complementary interengaging or interlocking members on an internal wall of the dial knob and external wall of the cartridge or housing. Such a rotation locking mechanism may be released by moving the dial knob against the force of the spring or resilient member whereupon a user may “dial” a dose of medicament by rotating the dial knob. After revolution or fraction of revolution of the dial knob, the user can release the knob whereupon it is returned back to its locked position by the action of the spring or resilient element. The rotation locking mechanism may therefore require a “pull and twist” action or a “push and twist” action to release and rotate the dial knob.

The deactivation mechanism may alternatively involve a decoupling of the drive member and dial knob. When the drive member and dial knob are decoupled, the dial knob is freely rotatable, that is free-spinning, but cannot actuate the drive member. For example, where the drive member comprises a threaded rod embedded within the medicament inside the cartridge, the dial knob may be moveable from a first, rest position free from the drive rod to a second, operable position coupled to the drive rod. Thus, in order to operate the applicator, the dial knob has first to be pushed in to couple with the drive rod and then rotated or dialed to advance the piston. Whilst requiring positive action to dispense medicament, the “push, engage and twist” action is easily performed without inconvenience to the user.

To guard against unwanted unwinding of the threaded drive member, the drive member and cartridge may be provided with complementary formations that co-operate with each other when the dial knob is in a coupled position to prevent counter-rotation of the drive member with respect to the cartridge. Effectively, the drive member may be axially locked with respect to the piston. For example, the drive member may comprise a base portion provided with a plurality of ribs extending radialy outwards therefrom and the cartridge wall is provided with a plurality of complementary ribs extending radialy inwards therefrom, wherein the dial knob splays the ribs of the drive member base portion outwards into overlapping relationship with the internal ribs on the cartridge wall upon coupling of the dial knob with the drive member.

In such an arrangement, the base portion of the drive member may comprise a collar on which the radialy extending ribs are carried. The collar is adapted to splay outwards under pressure exerted by the dial knob when the latter is brought into a coupled relationship with the drive member. For example, the collar may comprise a resilient material that can expand outwards under the pressure of the dial knob, or the collar may be provided with one or more longitudinal slits to permit splaying of the collar. The dial knob may comprise an internal ring concentric with, but spaced from, the outer dial knob wall for splaying the collar of the drive member when the dial knob is brought into coupling relationship with the drive member. Preferably the ring has a tapered external side wall from its leading edge to facilitate insertion of the ring in the collar of the drive member base portion and to gradually splay the collar as the taper is advanced in the collar. It will be appreciated that as the tapered portion of the side wall enters and progresses inside the collar, the ribs on the collar are forced between the ribs on the internal cartridge wall. In this way, counter-rotation of the drive member relative to the cartridge is substantially prevented.

It will be understood that when the drive rod is decoupled as described above, the drive rod itself “floats” freely within the cartridge. As well as preventing accidental dispensing of the ointment from the cartridge, this also allows for contraction and expansion of the ointment in the cartridge caused by changes in ambient temperature. When the applicator is used in hotter environments, the risk of expanding ointment escaping via the aperture or bursting through the piston or a piston seal is eliminated or at least substantially reduced.

Other arrangements for avoiding leakage of medicament due to thermal expansion or for containing leaked medicament are also contemplated. For example, where the applicator is provided with a cover, the cover may be shaped to provide a space between the applicator face and the underside of the cover, at least in the region adjacent to the aperture, and into which medicament may leak under expansion. With this arrangement, a user of the applicator simply wipes clean the applicator face to remove any leaked substance prior to dialing up a dose of medicament.

Another option is to provide an escape passage in the cartridge port adjacent the discharging end of the cartridge through which medicament can seep if undergoing expansion during storage. Such an escape passage will permit leaked medicament to be received within the applicator head, such as in an expansion chamber thereof, thereby avoiding any need for a user to clean the applicator face of leaked medicament prior to use, as required above. From time to time, the applicator head can be rinsed to clean any leaked medicament from within.

A further alternative solution for dealing with thermal expansion is to provide an expansion chamber in the piston, such as in a plunger thereof. The expansion chamber has a flow passage extending therefrom to the interior of the cartridge to permit medicament to expand into the expansion chamber. The volume of the expansion chamber will preferably equate to the maximum expansion volume of the medicament so that even when the cartridge is full, there is room to accommodate the expanded medicament. In this arrangement, a relatively small volume of medicament may remain in the cartridge, “trapped” in the expansion chamber, when apparently fully discharged. The amount of medicament that may remain in the chamber will typically not exceed about 1 g in a 50 g cartridge, so this is relatively small.

A particularly preferred solution for dealing with thermal expansion involves back-winding the dial knob. Thus the dial knob of the drive mechanism is preferably rotatable about a longitudinal axis of the applicator in a forwards direction for advancing the piston with respect to the drive member to dispense medicament and counter-rotatable (i.e. rotatable in the opposite direction) for advancing the drive member with respect to the piston. Such a backwind mechanism requires that the drive member has freedom to move, at least for a short distance, forwards and backwards along its longitudinal axis. Thus, the drive member and dial knob are preferably not integral components, rather the drive member and dial knob are coupled such as to permit the drive member reciprocal movement upon backwinding of the dial knob.

When the applicator has been fitted with a cartridge comprising a drive rod, it is preferred that the dial knob remains coupled with the end portion of the drive rod at all times. In this way, the user is assured that the dial knob is always operable on the drive rod, whether for dialing to dispense medicament or for back-winding for storage as desired. Accordingly, when the dial knob comprises a bore, spindle or the like, this has a length sufficient to retain the end of the drive rod therein irrespective of whether the drive rod is in a dispensing or back-wound position.

As will be understood, when the dial knob is counter-rotated, or back-wound, the drive member is advanced (i.e. it moves in the dispensing direction) while the piston remains stationary thereby creating expansion capacity behind the piston. In other words, the piston and the drive member can be forced backwards together under the pressure of expanding medicament as far as the distance advanced. In this way, thermal expansion of the medicament in the cartridge may conveniently be accommodated.

It will be appreciated that even when the applicator is provided with a valve and valve switch to close the aperture in the applicator face, some leakage of medicament may still occur if the pressure within the applicator rises due to thermal expansion. Hence it is recommended that the dial knob is always back-wound after use to guard against any such leakage.

If the dial knob is back-wound, it becomes necessary for a user to “forward wind” the dial knob to its original position before the next dose of medicament can be dispensed. Thus the dial knob should be forward rotated by the same degree as it is back-wound. For the sake of simplicity and to provide sufficient expansion capacity for reasonable temperature variations, the dial knob is preferably counter-rotated by a single revolution.

Back-winding by a single revolution is generally sufficient to provide expansion capacity to accommodate a temperature change of about 15° C. in the medicament. After such back-winding, the user is required to forward wind the dial knob by a single revolution prior to next using the applicator.

When the drive rod is not fixed in relation to the dial knob, for example to accommodate back-winding as described above, for the dial knob to be able to drive the piston forwards to dispense medicament, it is desirable to restrict the displacement of the drive rod in the reverse, longitudinal direction (i.e. movement away from the dispensing direction). Accordingly, the applicator preferably includes a stop for limiting the extent of longitudinal movement of the drive rod in the reverse direction. For example, the stop may comprise an upwardly directed stop surface, such as a projection, preferably a flange surrounding a bore in the dial knob, for abutting against a projection, which may also be in the form of a flange, provided on the drive rod behind the piston and spaced from the end of the rod. Thus when forward wound by the dial knob, such as would occur during priming of the applicator after fitting the cartridge or following back-winding, the drive rod is able to move down in a reverse direction only as far as the position where the drive rod flange or other projection abuts the stop surface around the dial knob bore. When linear movement of the drive rod is prevented, further forward rotation of the drive rod by the dial knob results in the plunger being advanced and medicament dispensed. If the dial knob is subsequently back-wound, the drive rod is driven upwards such that the flange on the drive rod moves away from the stop surface.

In a preferred arrangement, the stop surface for the drive rod is provided on the dial body of the dial assembly, for example, at the upper end of a tubular portion of the dial body that substantially surrounds the hollow spindle of the dial knob.

It is advantageous for a user to be able to determine whether or not the applicator is ready for dispensing or whether it is in storage mode, that is the when dial knob has been back-wound. Accordingly, the applicator preferably further includes a back-wind status indicator mechanism for indicating whether the applicator is in dispensing mode or storage mode.

Conveniently, the back-wind indicator mechanism is operable by means of the drive member, wherein a first, storage status is indicated when the drive member is advanced by back-winding of the dial knob and a second, dispensing status is indicated when the drive member is reversed by winding the dial knob forwards such that the applicator is primed and ready for use. Preferably the applicator displays the storage status when the drive member has been back-wound by 360° or more from its primed position.

The indicator mechanism is preferably housed within the dial assembly, most preferably within the dial knob. Accordingly, the status of the applicator may be conveniently displayed through an end face of the dial knob. For example, the indicator mechanism may comprise an indicator member that is pressed against the end face of the dial knob by the drive member when the applicator is primed and ready for use (i.e. as a result of forwards rotation of the dial knob) and is raised above the end face when the applicator is in the storage mode (i.e. as a result of the drive member being advanced by back-winding the dial knob through 360°).

The end face of the dial knob may be at least partially translucent or transparent such that a user can observe when the indicator member contacts the end face, indicating that the applicator is in the primed condition. The end face may comprise an end cap, such as a transparent end cap that has been rendered translucent, for example by back-printing or frosting.

When the indicator member is not in contact with the end face of the dial knob, it is preferred that end face displays a contrasting indication to that provided by the indicator member, thereby informing a user that the applicator is in the back-wound (storage) condition.

Advantageously, the end face of the dial knob comprises an end cap having a clear central lens ring on an inner surface thereof which lens ring reflects the colour of the surface proximate the lens ring onto the external face of the end face. The end cap is preferably translucent apart from the transparent central lens ring. By selecting different colours for the indicator member and the portion of the dial knob surrounding the end cap, the lens ring is able to reflect the different colours onto the external face of the end cap depending on the position of the indicator member.

In a particularly preferred arrangement, the dial knob comprises an outer dial ring, an inner spindle having a bore for receiving the end of the drive member, and an end cap provided on its inner surface with a transparent lens ring concentric with the bore, the end cap being substantially translucent except in the region of the lens ring, and the backwind indicator mechanism contained within the dial knob comprises an indicator member having an elongate portion extending into the bore and contactable by the end of the drive member, directly or by means of an indicator dowel or the like that sits in the elongate portion. and a ring portion concentric with and having a diameter greater than the lens ring. The dial knob, or at least a part thereof adjacent to the end cap, has a first colour and the indicator member, or at least a portion thereof, such as a ring portion, has a second, different colour. Thus when the indicator member, or the ring portion thereof, is pressed against the end face by the drive member acting on the elongate portion, the lens ring reflects the colour of the indicator onto the external face of the end cap and when the indicator member is raised by back-winding of the dial knob the lens ring reflects the colour of the dial knob adjacent the end cap onto the external end face.

Preferably the indicator member, or at least a ring portion thereof, is coloured green and the dial knob adjacent the end cap is coloured red. In this way, a green ring is reflected onto the external face of the end cap when the applicator is primed, ready for use and a red ring is reflected onto the external face when the applicator is in storage mode, after back-winding of the dial knob.

Of course, it will be appreciated that some persons may have difficulty in discerning between green and red, thus alternative colour schemes may be utilised. For example, the indicator ring may include castellated cut-outs, printed blocks or the like, such that a patterned ring is reflected onto the external end cap face. In this way, a person who may suffer colour-blindness should be able to discern a difference in the reflected ring as between primed (patterned ring) and storage (plain ring) states.

In order to raise the indicator member when the drive member is back-wound, the indicator mechanism preferably further includes an indicator spring interposed between the indicator member and the end face of the dial knob, whereby when the dial knob is forwards rotated the drive member presses the indicator member against the end face, and when the dial knob is counter-rotated or back-wound, the spring urges the indicator member away from the end face. Preferably the spring is compressed between the indicator member and end face when the applicator is primed by forwards rotation of the dial knob. Thus when the drive member advances upon back-winding, the spring lifts the indicator member up, away from the end face.

The applicator may include indicia to indicate to a user when a dose has been dispensed, such indicia may comprise a mechanism, such as a clicker mechanism, for producing audible feedback to a user. For example, an internal wall of the dial knob and an external wall of cartridge may be provided with complementary features that produce an audible indication, such as an audible click or multiple clicks, on each complete revolution or fraction of revolution of the dial knob.

More preferably, the clicker mechanism is provided on a dial assembly as hereinbefore described. For example, the dial assembly may comprise a dial body for engaging with applicator body and a dial knob rotatably connected to the dial body, the dial knob carrying one or more clicker arms and the dial body having one ore more clicker posts, wherein the clicker arm(s) ride over the clicker post(s) on rotation of the dial knob to produce an audible click. The dial knob preferably has two clicker arms and the dial body preferably has four equally spaced clicker posts so that an arm rides over a post every 45° of rotation of the dial knob in either direction. As will be appreciated, a predetermined amount, such as 0.1 g, of medicament is dispensed per click.

An audible indication may otherwise be achieved by means of a ratchet mechanism. Alternatively or in addition thereto, the dial knob may have a visible indicator, such as on an external side wall, that aligns with a visible indicator on the cartridge. The indicators are aligned at the commencement of rotation of the drive knob, moving out of alignment during rotation, and back into alignment after a complete revolution of the dial knob. The cartridge may also include further or “part revolution” indicators, for example, at quarter, half and three-quarter positions from the primary indicator so that a user can determine when a fraction of a “full revolution” dose has been dispensed.

Usefully, the applicator also includes a fill indicator for providing a user with information on how much medicament remains in or has been dispensed from the cartridge.

The fill indicator may comprise a counter mechanism, preferably linked with the dial knob whereby each revolution of the dial knob advances the counter mechanism by a unit. The counter mechanism may comprise an incremental scale, visible to a user, to indicate the number of doses or volume of medicament dispensed or remaining, or to provide a general indication of usage, particularly to indicate when the cartridge is approaching empty.

In one arrangement, the counter mechanism comprises a rotatable disc viewable through an aperture the base of the dial knob, the disc carrying a circular display, either of doses delivered or remaining, or volume dispensed or remaining.

In another arrangement, the counter mechanism may comprise a gear mechanism that translates a rotation of the dial knob into an intermittent rotary motion, such as a harmonic drive mechanism, or a Geneva wheel mechanism. The latter mechanism, most preferably in the form of a twin Geneva wheel mechanism, is preferred. In a twin Geneva mechanism, a single rotation of the dial knob results in the advancement of a driven wheel by one step. Thus a marker on the driven wheel may be tracked against a dosing counter scale provided on the outer wall of the cartridge or housing therefor adjacent the dial knob.

Since it is frequently inadvisable to dispense more than a predetermined amount of medicament daily, for example a patient should not administer more than about 15 g of psoriasis ointment each day, any counter mechanism is preferably re-settable. Thus each day a user may re-set the counter mechanism to zero prior to dispensing a first dose of ointment. Of course, by re-setting the counter mechanism, a user may have little indication of how much ointment has been dispensed in total, or how much remains to be dispensed.

Accordingly, instead of linking the fill indicator with the dial knob or in addition thereto, an indicator may be linked with the piston whereby the position of the piston can be detected or observed. As will be appreciated, the piston advances up the cartridge or housing along the drive member to dispense medicament. Thus, in its simplest form, the dial knob may be provided with an aperture through an end surface thereof and through which a user may view the position of the piston.

Other means of tracking the advancement of the piston to determine the fill level of the cartridge may be used. For example, the piston may be provided with a blade that scratches the internal wall of the cartridge as the piston rises. Most conveniently, such a blade may extend laterally from the trailing edge of the piston.

In order for a user to observe the extent of the score mark made by the blade, it is desirable that the cartridge be manufactured from a transparent, semi-transparent or translucent material. To provide a highly visible indicator, the internal wall of the cartridge, at least in the vicinity of the member of the blade, may be coated with a material that is scraped off or delaminated under the action of the blade. In the instance where the cartridge is retained inside a housing, the housing may comprise an elongate window or aperture through which a user can view progress of the score mark as the piston and blade advance up the cartridge. The cartridge and/or the housing may also carry a graduated scale, adjacent the blade member, so a user can determine the volume of medicament used or remaining.

Another means of tracking advancement of the piston comprises a ribbon having a first end attached to the piston, preferably attached to the trailing end of the piston, and extending around the base of the cartridge and against the external cartridge wall, terminating in a second, free end. Prior to dispensing any ointment, the ribbon will extend from behind the piston for substantially the full length of the external cartridge wall. As medicament is dispensed, the piston advances up the cartridge and drags the ribbon behind it. Thus the second end of the ribbon will be gradually drawn down the external wall thereby to provide an indication of how much the cartridge has been depleted. The ribbon may be held loosely against the external cartridge wall by means of an elongate enclosure, preferably transparent. If the cartridge is retained in use inside a housing, the housing preferably includes an elongate window through which the ribbon may be viewed.

Whilst in general it is preferred that the applicator according to the invention be re-usable, namely to be used with replaceable cartridges, it is also contemplated that the applicator comprises a pre-filled disposable device, wherein the applicator head, drive mechanism and cartridge are all disposable, for example as a single unit, once the ointment has been fully discharged or when a user has completed a course of treatment. In such a disposable applicator, the cartridge is preferably preassembled with the applicator head, most preferably with an external housing to protect the cartridge from being inadvertently “squeezed” or otherwise deformed and ointment being discharged in a random manner.

In relation to a disposable applicator or a re-usable applicator, the applicator head is preferably integral with an applicator housing, typically of moulded plastic, and wherein the housing has an open end opposite the applicator head end where a cartridge can be inserted. After a cartridge has been inserted, the housing open end may then be closed by means of the dial knob, preferably as part of a dial assembly that connects with the applicator housing, and which engages with the drive member at the base of the cartridge. The applicator head is supplied for use typically with a removable cover as described above.

When disposable, the applicator can easily be maintained in a hygienic condition for the duration of its life, being little risk of any microbial contamination, especially in the region of the sealable aperture.

The applicator according to the invention may equally comprise a re-usable applicator head, wherein one cartridge can be withdrawn from the cartridge port and a second or further cartridge fitted. When the applicator head includes, for example, a metal applicator face, the ability to re-use it makes sense economically and from an environmental perspective.

It is desirable that a re-usable applicator head is washable and/or is able to tolerate cleaning with anti-bacterial or anti-microbial agents. While in general the applicator head comprises a readily-cleanable surface, users of the applicator may wish to conduct a more thorough cleansing of the head from time to time.

When the applicator includes a housing for encasing the cartridge, this too is preferably re-usable along with a re-usable head. Thus, a user may retain one of more sets of applicator heads and housings for use with replaceable cartridges, such as cartridges of different volumes. In this regard, the dial knob of the applicator may be connected or connectable to, and form part of, the housing, such as by connection to a dial body as part of a dial assembly which in turn is connectable to an applicator body (the dial body and applicator body together forming a cartridge housing). Again, the housing is also preferably washable and/or cleansable using anti-microbial/anti-bacterial agents. The dial knob may also be detachable for cleaning.

It will be understood that the present invention resides not only in an applicator as hereinbefore described for use with a medicament cartridge, but also in such an applicator when fitted with a medicament cartridge. Moreover, the invention further resides in a medicament cartridge for use with the applicator.

Thus, from another aspect, the invention also comprises a cartridge for use with an applicator as hereinbefore described. Preferably the cartridge is a pre-filled cartridge, being pre-filled with medicament, particularly but not limited to medicament for the treatment of psoriasis.

As previously described, the cartridge may comprise a flexible bag, such as of foil, having a first, discharging end adapted to co-operate with a cartridge port of a medicament applicator and a second end, wherein the bag is compressible under pressure exerted against the second end by a piston to force medicament charged to the cartridge out through the discharging end. The discharging end of the flexible bag may comprise a substantially rigid end cap, preferably having an external thread, bayonet fixing or the like, which engages with the applicator head via the cartridge port. The end cap may be sealed with a closure member, such as a foil cover, that can be peeled off before connecting the bag to the applicator head or punctured by the cartridge port on connection. The flexible bag may be substantially tubular, preferably for fitting within a tubular housing engageable with the applicator head, or an applicator body integral with the applicator head, of the applicator.

The flexible bag may alternatively be provided with an elongate bore, extending from an opening at the second end for accommodating a drive member, such as a threaded drive rod. The bore terminates close to, but spaced from, the discharging end to provide a continuous pathway for medicament to reach the discharging end and be dispensed as the bag is compressed under pressure exerted by the piston acting against the second end as it advances along the rod.

More preferably the cartridge comprises an elongate container, such as a tube, charged with medicament, the container having a first, discharging end for co-operating with a cartridge port of a medicament applicator according to a first aspect of the invention and a second end, a drive rod extending longitudinally within the container and a piston mounted on the drive rod and in sealing contact with the container inner wall for advancing along the container to dispense medicament from the discharging end upon rotation of the drive rod in a forwards direction by the applicator dial knob, wherein the drive rod includes an end portion projecting beyond the piston, the end portion being adapted for coupling with the dial knob of the applicator whereby the drive rod is rotationally fixed relative to the dial knob so that the drive rod is rotated with the dial knob and is permitted relative axial movement relative to the dial knob shaft to raise or lower the drive rod with respect to the dial knob during priming and back-winding operations.

The projecting end of the drive rod of the cartridge is preferably adapted to be received in a bore of the dial knob. Moreover, the projecting end ideally has a complementary profile to that of the bore, for example in the form of a polygonal cross-section or radial projections. Most preferably the end portion of the drive rod is splined for interlocking with longitudinal grooves in dial knob bore. The end portion may be formed integrally with the drive rod, or may be connected thereto.

Preferably the drive rod is threaded substantially along its length, except in the region of the projecting end portion. More preferably, the drive rod is provided with a lateral projection, such as a flange, for abutment with a stop surface on the applicator, such as may be provided on the dial knob or dial body of a dial assembly, thereby to restrict linear movement of the drive rod in a direction away from the dispensing end and cause the piston to rise on the drive rod during forwards rotation of the dial knob to dispense medicament. The lateral projection is ideally provided on the drive rod between the threaded portion and the projecting end portion.

The cartridge is preferably supplied for use with the flange in contact with the rear, trailing face of the piston, effectively providing a shelf-life seal at the non-dispensing end of the cartridge. Once the cartridge is fitted to the applicator and the dial knob rotated to prime the applicator, the flange will of course move away from the piston as the drive rod is backed down.

The cartridge tube preferably terminates at its second end with an outwardly directed support flange which acts as a base and enables the cartridge to be stood in an upright position, for example, during storage.

The tube wall of the cartridge preferably extends sufficiently to accommodate the projecting end of the drive rod for protecting the end against possible damage prior to fitting in the applicator. In other words, the second end of the cartridge is substantially hollow. Advantageously, the tube wall of the cartridge extends for a distance beyond the tip of the projecting end, sufficient to accommodate any movement of the drive rod resulting from thermal expansion of the medicament in the cartridge during storage, that is prior to fitting the cartridge in the applicator.

Thermal expansion of medicament may remain an issue even after fitting of the cartridge in the applicator. In order to avoid any leakage from the cartridge due to thermal expansion, the cartridge is adapted to permit reciprocal movement of the drive rod such as to allow the drive rod to be advanced in the cartridge by back-winding of the dial knob whilst the piston is held stationary. Accordingly, the length of the drive member is preferably selected such that it extends substantially along the length of the cartridge thereby enabling substantially all of the medicament to be discharged, whilst at the same time accommodating reciprocal movement achieved by back-winding the applicator dial knob at least one revolution.

In a particularly preferred arrangement, the projecting end portion of the drive rod is adapted to be operable upon a backwind indicator mechanism of the applicator. In particular, the drive rod end portion may be operable on an indicator member of the mechanism. For example, when the drive rod is advanced, such as when the applicator is primed for use or is dispensing a dose of medicament, the end portion acts on an indicator member to press the member against the end face of the applicator dial knob.

When the drive rod is reversed or backwound, the end portion permits the indicator member to rise above the end face.

The end portion of the drive rod may have a substantially flat end face for acting on an elongate portion of the indicator member. Alternatively, the end face of the end portion may be concave or convex for co-operating with a complementary end face of the indicator member.

The piston housed within the cartridge preferably comprises a resilient material for sealing around the threaded drive rod and exerting a sealing force against the internal tube walls. The dimensions and resilience of the piston are selected so that the frictional force exerted by the piston against the tube walls outweighs the frictional force between the threads of the piston and drive rod, such that upon rotation of the drive rod the piston is prevented from rotating relative to the walls but instead advances along the thread of the drive rod.

Advantageously, the lead face of the piston comprises an inclined shoulder portion for matching an inclined shoulder portion at the discharging end of the cartridge. Having such a complementary shoulder profile ensures that the piston is able to expel all the medicament from the tube and thereby avoid waste. It also means that used cartridges can be disposed of safely at home and not as medical waste.

The piston may comprise a unitary body of resilient material, but more preferably comprises a leading drive nut, a drive seal abutting against the drive nut for sealing around the thread of the drive rod behind the drive nut, and a resilient plug surrounding the drive nut for exerting a sealing force against the internal tube walls.

The drive seal guards against loss of medicament in the threads of the drive rod and helps maintain the cartridge clean, especially where the drive rod becomes exposed behind the piston as the medicament is dispensed.

Preferably the piston, or the plug thereof, is provided on an outer circumference with one or more seals, such as lip seals or O-ring seals, to provide the seal against the internal tube wall of the cartridge. Provision of such seals facilitates smooth advancement of the piston up the cartridge tube. The seals are preferably elastomeric and may be formed integrally with the piston, for example, by means of a two-shot moulding process. Such a process may involve a first moulding shot using a relatively rigid material followed by a second moulding shot using an elastomeric material to form the seals. The core of the piston or plug may also incorporate a seal to seal against the drive member.

While the drive rod preferably extends centrally along the cartridge tube so that the rod, piston and tube are concentric with respect to each other, the drive rod may alternatively be offset with respect to the tube and piston thereby to provide an eccentric drive mechanism. In the latter case, asymmetric forces between the piston and the tube wall serve to drive the piston along the tube.

In a threaded drive rod, the pitch of the drive rod thread in the cartridge is preferably selected such that one complete revolution of rod (achieved for example by a single revolution of the dial knob) drives the piston along the drive rod by the required distance to expel a volume of medicament equivalent to a “single” dose. By “single” dose, it is meant a typical unit dose, for example 1.0 g, and in which case, a half-revolution of the dial knob will expel 0.5 g.

Typically, the cartridge tube is charged with medicament by filling from the second end and a drive rod and piston are inserted in the tube after filling, effectively sealing the second end. Any air trapped in the cartridge when the second end is sealed by the piston can result in the medicament oozing out through the valve aperture after a dose has been dispensed, thus it is desirable to ensure that no air remains trapped in the cartridge when the piston is fitted. To this end, the cartridge tube may have an enlarged bore, or counterbore, adjacent its second end for permitting air to escape as the piston is inserted and/or to allow excess medicament to squirt out in the event of overfilling.

More preferably, the tube wall is provided with at least one, and preferably a plurality, of longitudinally extending internal channels at the second end. The channel or channels act as air vents allowing air to escape around the piston seal(s) during assembly. In order not to compromise the cartridge's integrity, the tube wall may have external projections corresponding to the internal channels for maintaining a substantially constant wall thickness around the air vent features. The channels may be in the form of a series of grooves.

Alternatively, or in addition thereto, the drive rod may be provided with an elongate groove towards the end near the piston to provide an air bypass passage.

The discharging end of the cartridge, whether in the form of a bag or rigid tube, preferably comprises a shoulder portion, more preferably an inclined shoulder portion, having one or more interlocking features for securing the cartridge against the cartridge port of the applicator when fully inserted. The interlocking or engagement features for securing the cartridge in the applicator are most preferably formed, such as by molding, integrally with the cartridge tube.

Preferably the engagement mechanism is in the form of a bayonet mount, for example, comprising a pair of opposing outwardly directed male connectors, such as a pair of lugs, on the cartridge for insertion in corresponding slots of the applicator head, typically adjacent the cartridge port. The male connectors may be carried on an upwardly directed ring on a shoulder of the cartridge and concentric with the tube wall and cartridge nozzle. Following insertion of the lugs into the slots on the applicator, the cartridge may be rotated so that the lugs become trapped in complementary slots on the applicator head.

The cartridge preferably also comprises a neck portion extending from the shoulder portion, the neck portion terminating in an outlet from the cartridge for the medicament. Advantageously the neck portion has a length such that when the cartridge is fitted to the applicator the outlet lies substantially adjacent to an outlet valve on the applicator head. The neck portion may have an external screw thread for engagement with a sealing cap that is removed from the cartridge prior to fitting.

The leading edge of the neck portion may be provided with a face seal for sealing against an inwardly directed flange on the cartridge port when the cartridge is screwed onto the applicator head. The face seal effectively prevents medicament from penetrating between the cartridge and cartridge port during dispensing; this helps ensure that all medicament forced from the cartridge is directed towards the aperture and none is “lost” internally. Thus the presence of the face seal contributes to accuracy of dosing as well as helping maintain internal surfaces of the applicator free from extraneous medicament.

The neck portion of the cartridge may be a friction fit with a complementary neck portion of the cartridge port. In this arrangement, the neck portion of the cartridge may be provided with an O-ring or similar elastomeric seal around its outer circumference for sealing against a complementary neck portion of the cartridge port. An O-ring or similar elastomeric seal similarly prevents medicament from penetrating between the cartridge and cartridge port during dispensing. An O-ring seal or the like may alternatively be provided on the cartridge port.

The cartridges according to the further aspect of the invention may be supplied in a range of capacities and sizes, for example, containing 30 g, 60 g or 120 g or even larger amounts of medicament, but compact cartridges containing 5 g, 10 g or 15 g are also contemplated. Typically, the capacity is selected to provide sufficient doses to last a user one week or more.

Optionally, the cartridges may all share the same length but have different diameters so, for example, a 30 g cartridge may be have a length equal to that of a 60 g cartridge but of narrower diameter. It will be appreciated that with such an arrangement, the volume of medicament dispensed per revolution would differ, assuming the pitch of the threaded rod remained constant. For this reason, it is desirable that any counter mechanism provided with the applicator be re-settable for each new cartridge.

Typically, to maximise comfort for the user, a cartridge or, if used, a housing for the cartridge has a diameter in the range of from about 10 mm to 40 mm, more preferably about 15 mm to 35 mm, especially about 30 mm. For example, a 30 g or 60 g cartridge may have a diameter of about 30 mm, whereas a 120 g cartridge may have a diameter of about 35 mm.

Optionally, the cartridges may be supplied with the same dimensions, but filled with different volumes. For example, a 30 g cartridge may share the same dimensions as a 60 g cartridge but be filled to only half its capacity.

Irrespective of capacity, it is preferred that the cartridges have a universal configuration at the discharging end, so that they may be used interchangeably with the same applicator head. When the dial knob is not provided on the cartridge, the cartridges preferably also share a universal configuration at the opposite, drive end, so that they are all capable of co-operating with the applicator dial knob or dial assembly to discharge medicament.

In order that a user may store the applicator easily when not in use, the applicator is preferably provided with a stand having a hollowed recess of complementary dimensions to the applicator head and in which the applicator head may be nested. The applicator is therefore stored with the applicator face facing downwards in the stand and therefore is not exposed to dirt, dust or other potential contaminants. More preferably, where the applicator face is inclined with respect to the longitudinal axis of the applicator, the hollowed recess of the stand is also inclined so that the applicator housing or cartridge rests substantially vertically when the applicator is located on the stand.

The invention further resides in an applicator kit comprising an applicator as hereinbefore described, one or more cartridges therefor, and a patient information leaflet providing operating instructions for a user to fit the cartridge to the applicator and to then operate the applicator to deliver the desired medicament dosage. The leaflet further provides information on replacement of cartridges.

While the cartridges for use with the applicator head may be provided with a universal fitting, so that cartridges of varying capacity are able to fit onto the same applicator head, the kit may nonetheless include more than one applicator head, for example, having a different diameter for use depending on the area of skin being treated.

From another aspect, the invention resides in a method of applying a dose of semi-solid medicament to the skin of a user, said method comprising the steps (i) fitting a medicament cartridge as hereinbefore described to the cartridge port of an applicator also as hereinbefore described and coupling the drive rod of the cartridge with the dial knob, (ii) rotating the dial knob in a forwards direction to prime the applicator, (iii) further rotating the dial knob in a forwards direction by a complete revolution, or multiple or fraction thereof, to dispense a dose of medicament through the aperture onto the applicator face and (iv) applying the medicament dispensed onto the face of the applicator to the skin and spreading the medicament over the skin area to be treated by means of the applicator face.

Preferably the method further comprising the step (v) rotating the dial knob in a reverse direction to raise the drive rod and place the applicator in a storage mode.

Where the applicator of the invention includes a valve switch as hereinbefore described, the method advantageously involves, prior to step (iii), moving a valve switch to open the aperture on the applicator face and, prior to step (iv), moving the valve switch to close the aperture.

The invention will now be described in more detail, by way of example only, with reference to the accompanying drawings, all of which illustrate aspects of the invention as hereinbefore described:

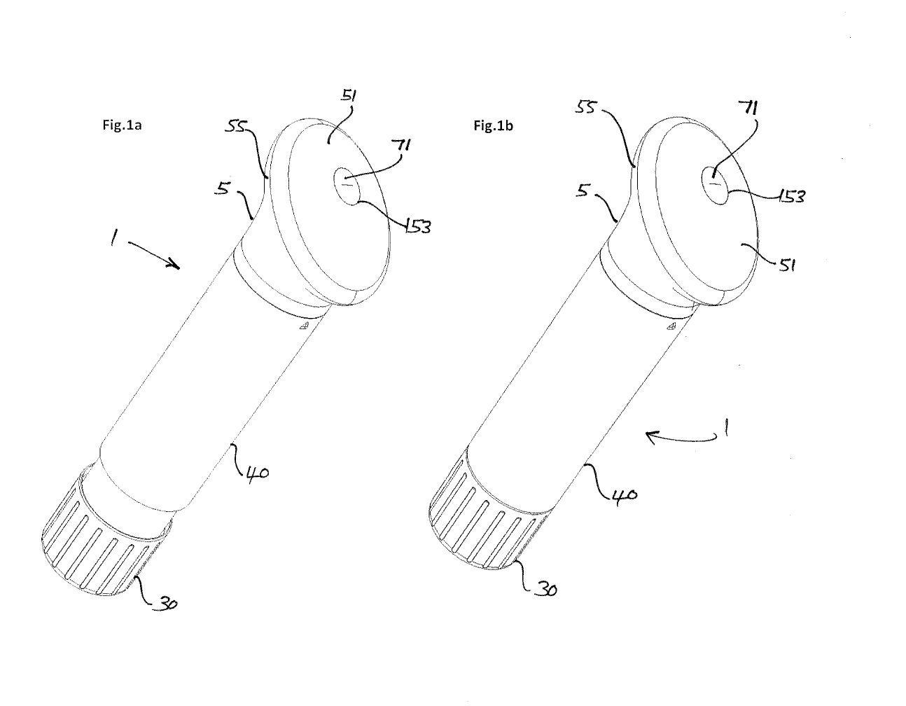

FIG. 1a is a perspective view of an applicator fitted with dial knob in an inoperative position;

FIG. 1b is a perspective view of the applicator of FIG. 1a with dial knob in an engaged position;

FIG. 1c is a view of a replaceable cartridge for use with the applicators of FIGS. 1a and 1b;

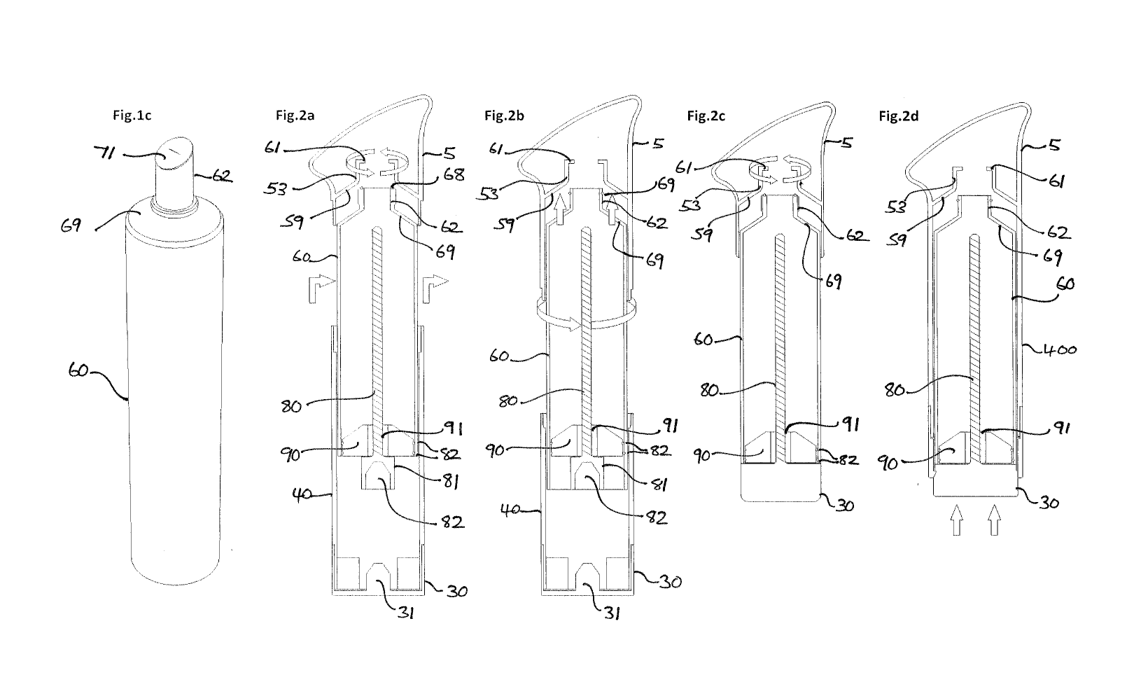

FIG. 2a shows a cross-section through an applicator provided with bayonet fitting, housing and a replaceable cartridge;

FIG. 2b shows a cross-section through an applicator provided with a screw fitting, housing and a replaceable cartridge;

FIG. 2c shows a cross-section through an applicator provided with a screw fitting and screw-on replaceable cartridge;

FIG. 2d shows a cross-section through an applicator provided with a replaceable push-in cartridge;

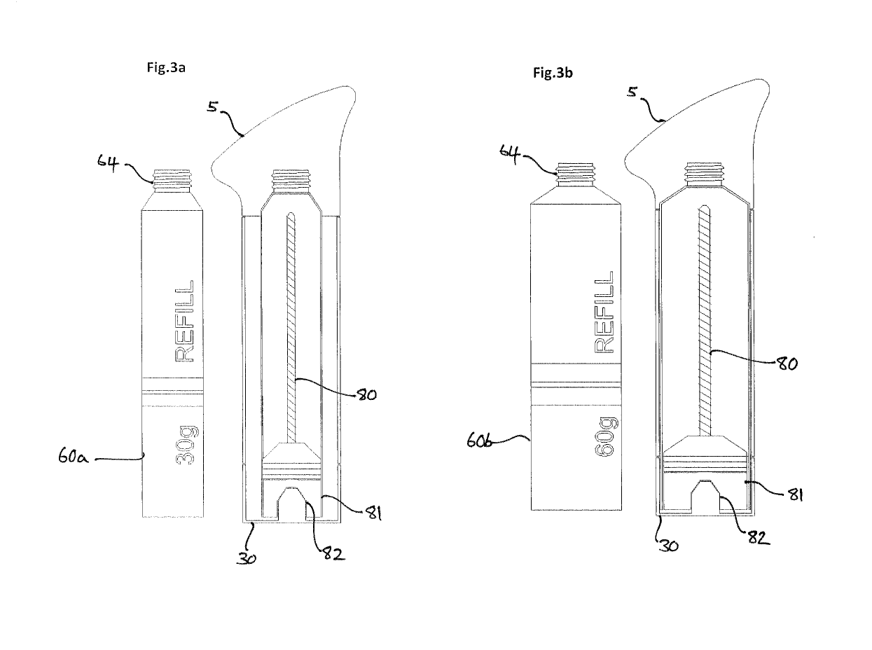

FIG. 3a shows a cross-section through an applicator fitted with a replaceable cartridge of a first size and the cartridge;

FIG. 3b shows a cross-section through the applicator of FIG. 3a fitted with a replaceable cartridge of a second size and the cartridge;

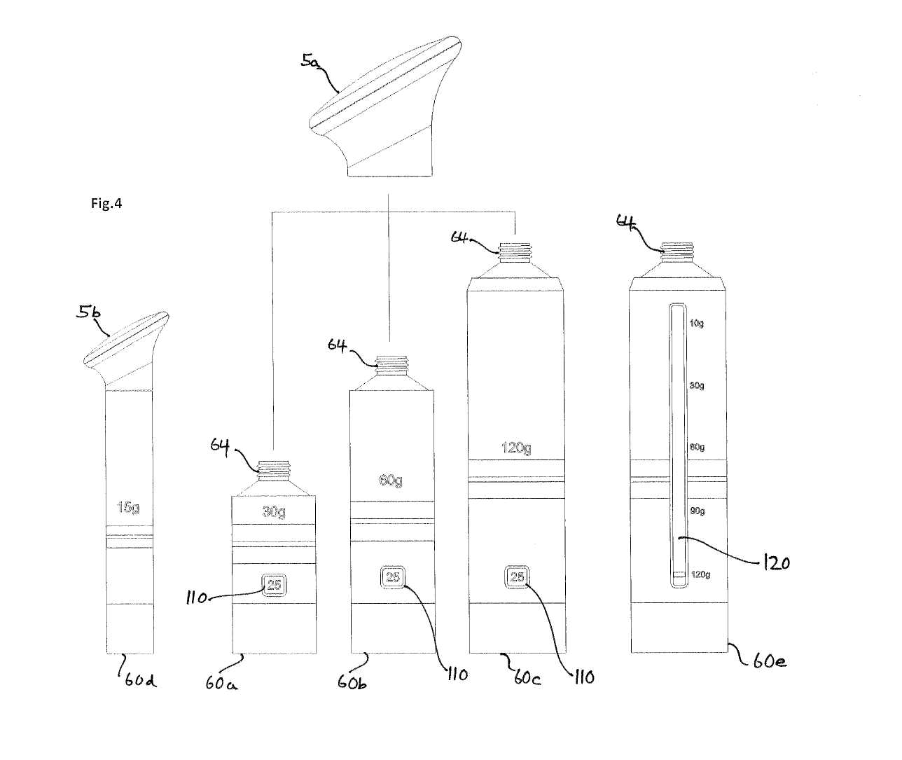

FIG. 4 shows a range of re-usable applicator heads and a range of disposable cartridges of varying volumes;

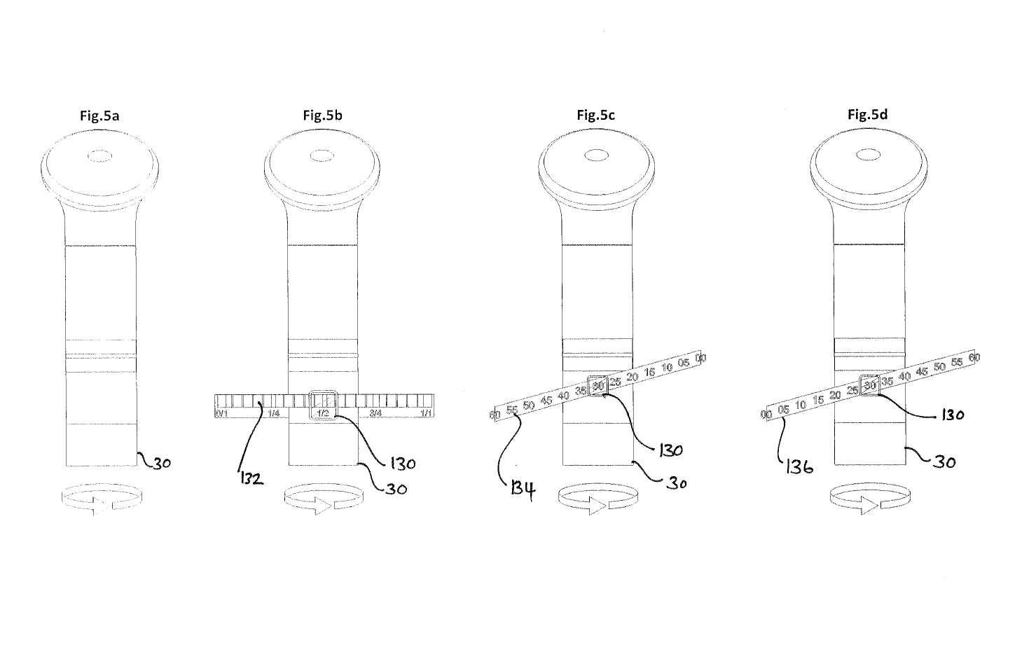

FIG. 5a shows an applicator having an audible counter mechanism;

FIG. 5b shows an applicator having a full/empty gauge;

FIG. 5c shows an applicator having a counter for dispensed doses;

FIG. 5d shows an applicator having a counter for doses remaining;

FIG. 5e shows an applicator having an automatically re-setting dose counter;

FIG. 5f shows an applicator having a digital dose counter;

FIG. 5g shows an applicator having a full/empty indicator;

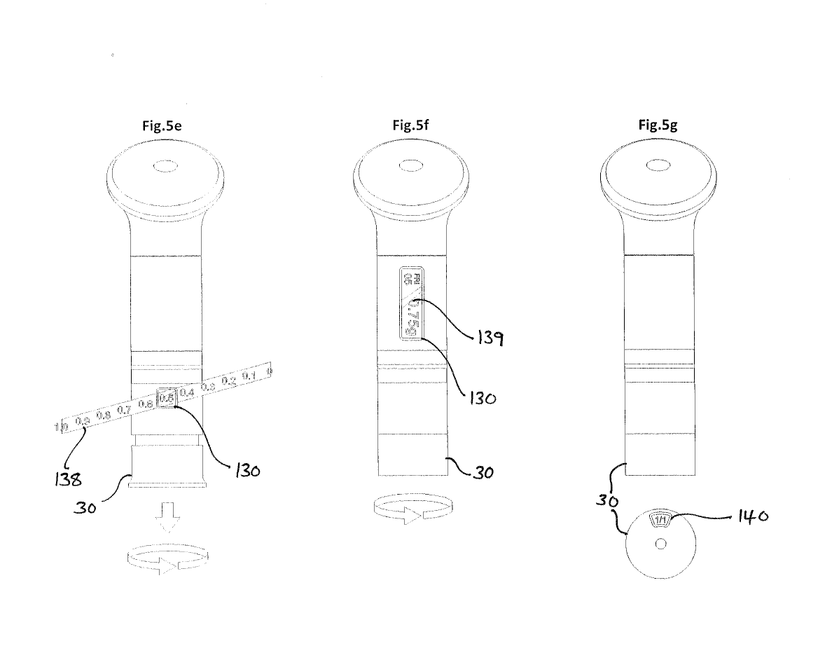

FIG. 6a shows an applicator having a basic fill indicator;

FIG. 6b shows an applicator having a ring drive counter mechanism;

FIG. 6c shows an applicator having a counter indicator;

FIG. 6d shows an applicator having a ribbon fill indicator;

FIG. 6e shows an applicator having a Geneva wheel indicator;

FIG. 6f shows an applicator having a harmonic drive indicator;

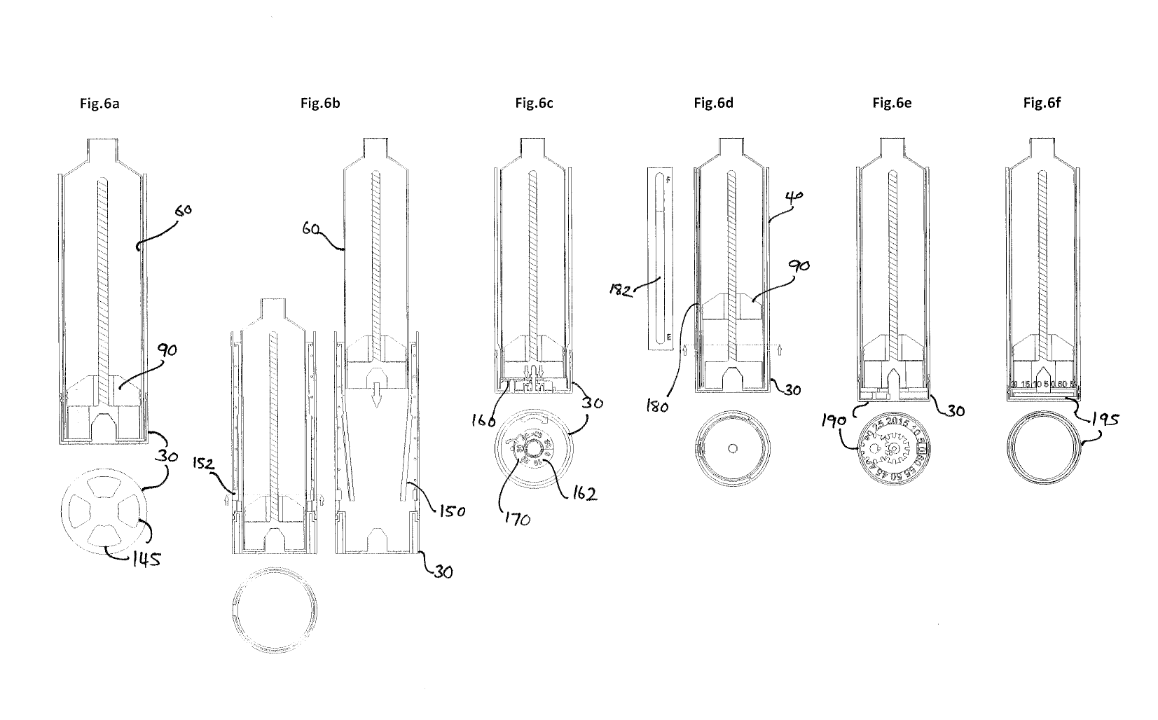

FIG. 7a shows an applicator having a scratch fill indicator;

FIG. 7b is a cross-section through the applicator of FIG. 7a;

FIG. 7c is a magnified cross-section through the scratch mechanism of FIG. 7b;

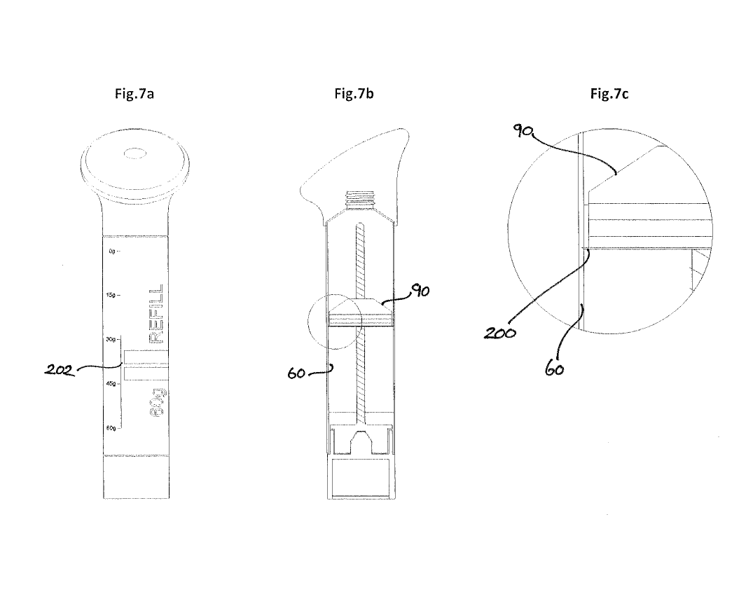

FIG. 8 is a cross-section through an applicator with an advanced piston construction;

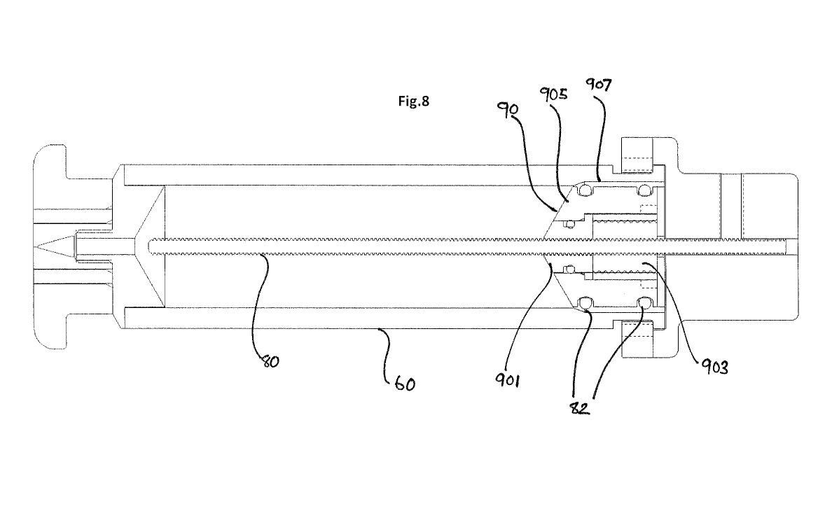

FIG. 9a is a side view of a filled, flexible cartridge;

FIG. 9b is a cross-section through the flexible cartridge of FIG. 9a;

FIG. 9c is a side view of an applicator for housing the cartridge of FIG. 9a;

FIG. 9d is a cross-section through the applicator of FIG. 9c containing the filled flexible cartridge of FIG. 9a;

FIG. 9e is a cross-section through the applicator of FIG. 9c containing a partially discharged flexible cartridge of FIG. 9a;

FIG. 9f is a cross-section through the applicator of FIG. 9c containing a fully discharged flexible cartridge of FIG. 9a;

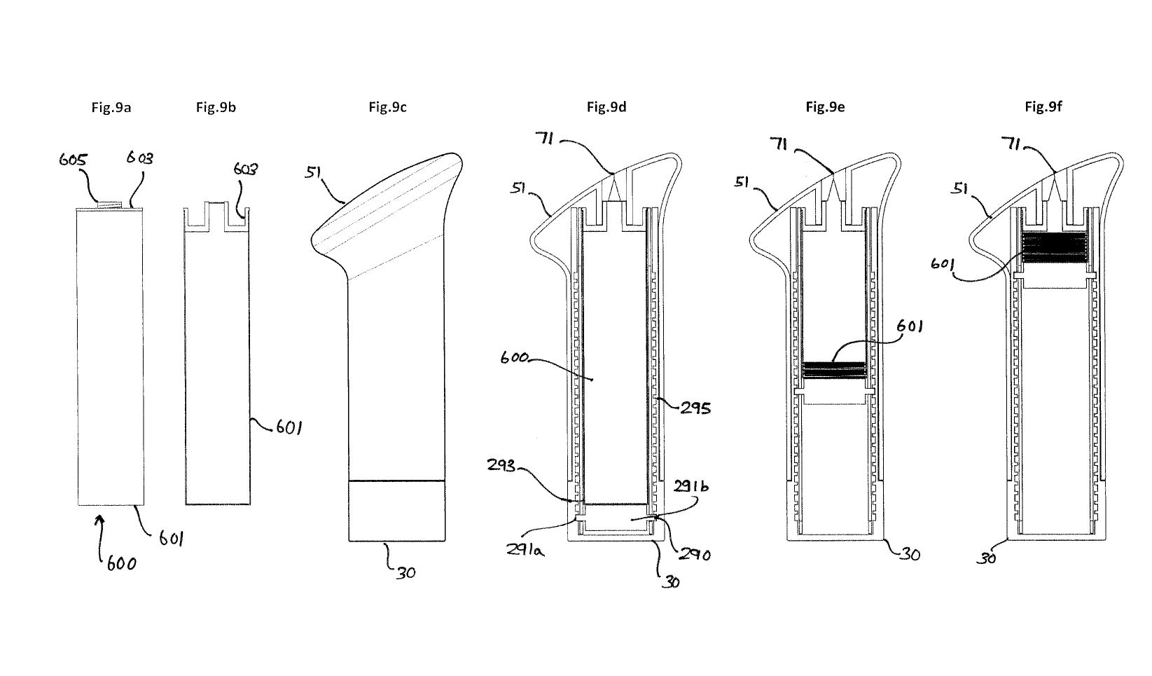

FIG. 10a is a side view of an alternative filled, flexible cartridge;

FIG. 10b is a horizontal cross-section through the flexible cartridge of FIG. 10a;

FIG. 10c is a vertical cross-section through the flexible cartridge of FIG. 10a;

FIG. 10d is a side view of an applicator for housing the cartridge of FIG. 10a;

FIG. 10e is a cross-section through the applicator of FIG. 10d containing the filled flexible cartridge of FIG. 10a;

FIG. 10f is a cross-section through the applicator of FIG. 10d containing a partially discharged flexible cartridge of FIG. 10a;

FIG. 10g is a cross-section through the applicator of FIG. 10d containing a fully discharged flexible cartridge of FIG. 10a;

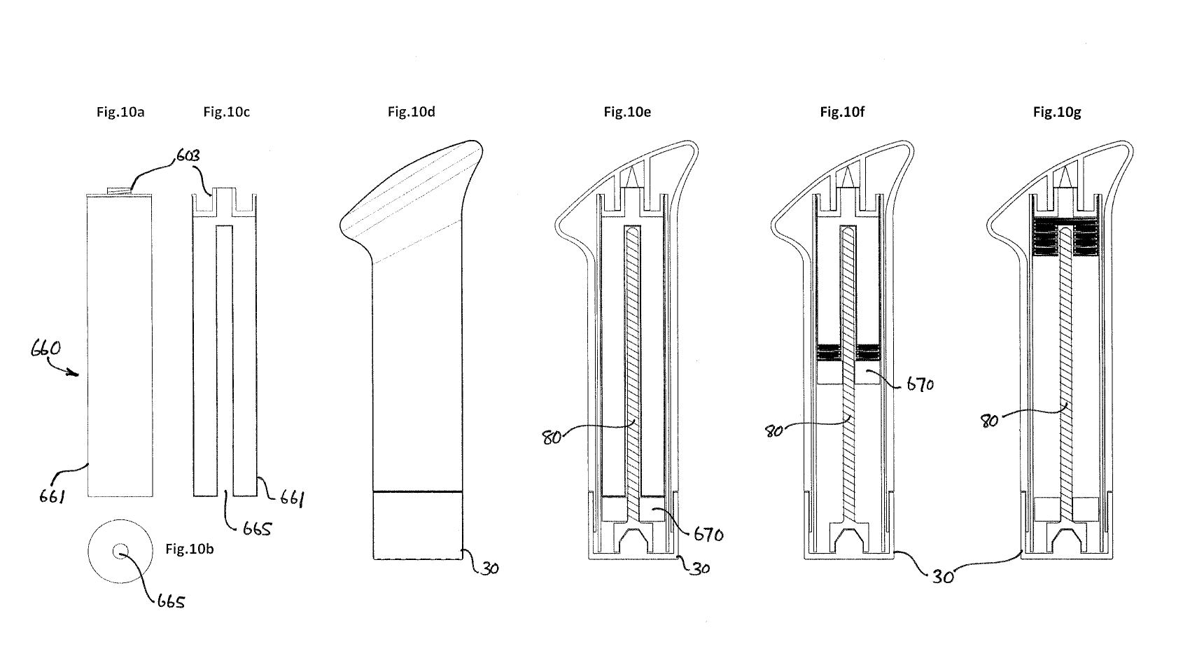

FIG. 11a is a cross-section through a sealed pre-filled cartridge at ambient temperature (25° C.);

FIG. 11b is a cross-section through the cartridge of FIG. 11a inserted in an applicator at raised temperature (40° C.) with dial knob disengaged;

FIG. 11c is a cross-section through the cartridge and applicator of FIG. 11b at raised temperature (40°) with dial knob engaged;

FIG. 11d is a cross-section through the cartridge and applicator of FIG. 11b at lowered temperature (0°) with dial knob disengaged;

FIG. 11e is a cross-section through the cartridge and applicator of FIG. 11b at lowered temperature (0°) with dial knob engaged;

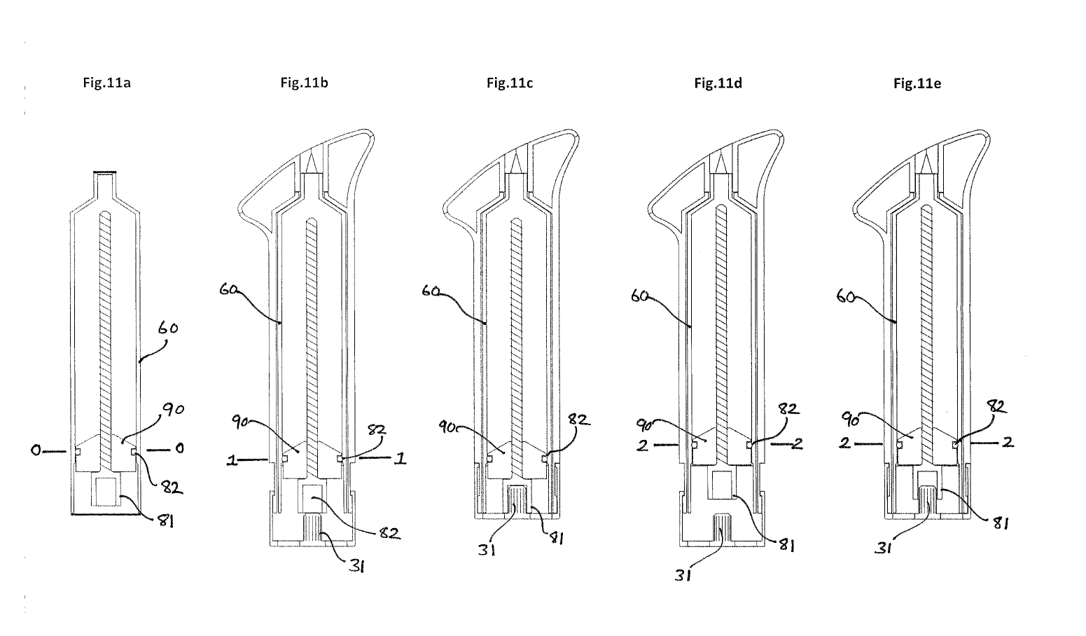

FIG. 12a is a cross-section through an applicator head fitted with a pin-valve cartridge with the aperture sealed;

FIG. 12b is a cross-section through the applicator head and cartridge of FIG. 12a with the aperture open;

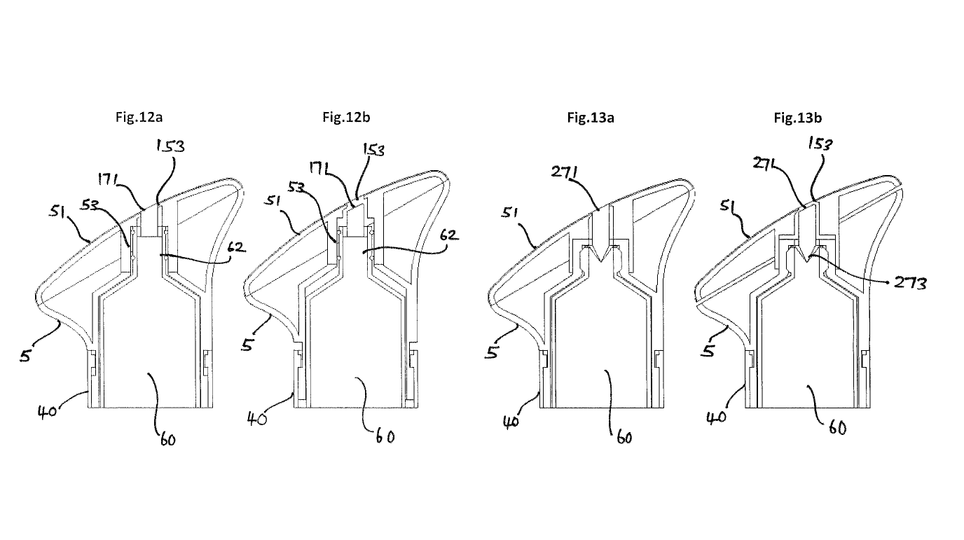

FIG. 13a is a cross-section through an applicator head with pin valve and a cartridge with the aperture sealed;

FIG. 13b is a cross-section through the applicator head and cartridge of FIG. 13a with the aperture open;

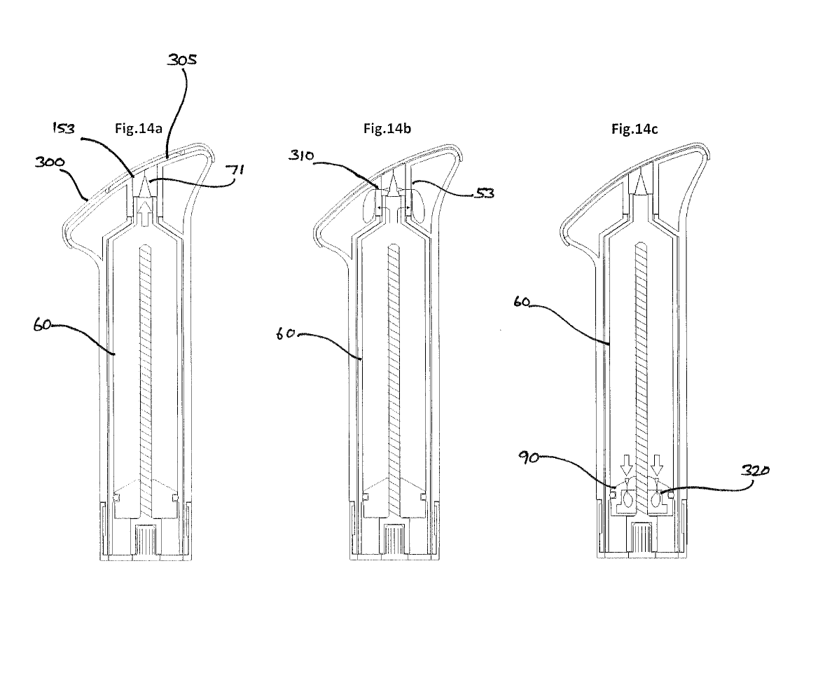

FIG. 14a is a cross-section through an applicator with removable cover;

FIG. 14b is a cross-section through an applicator adapted for thermal expansion;

FIG. 14c is a cross-section through an alternative applicator adapted for thermal expansion;

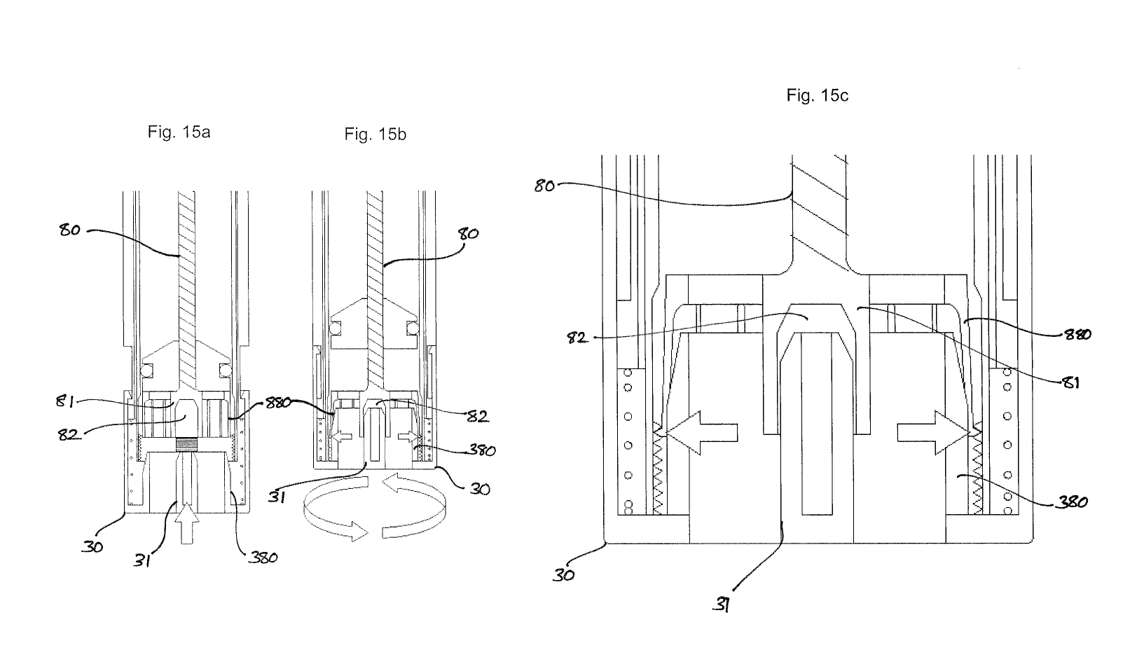

FIG. 15a is a partial cross-section through an applicator with a dial knob in a deactivated position;

FIG. 15b is a partial cross-section through the applicator of FIG. 15b with the dial knob in an activated position;

FIG. 15c is a magnified cross-section showing the coupled dial knob and drive member of FIG. 15b;

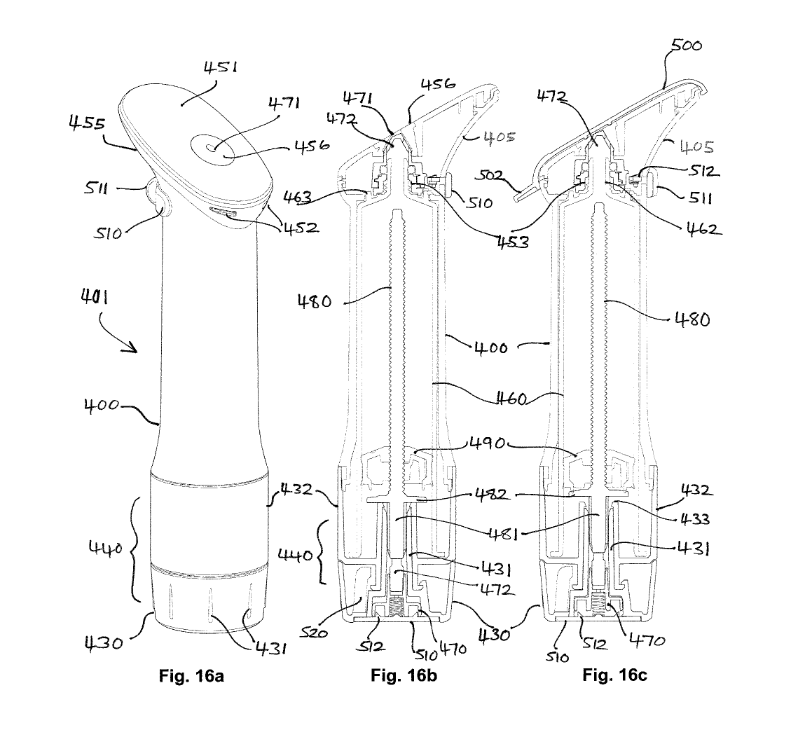

FIG. 16a is perspective side view of an applicator with a valve switch;

FIG. 16b is a cross-section through the applicator FIG. 16a primed ready for use;

FIG. 16c is a cross-section through the applicator of FIG. 16a in storage mode;

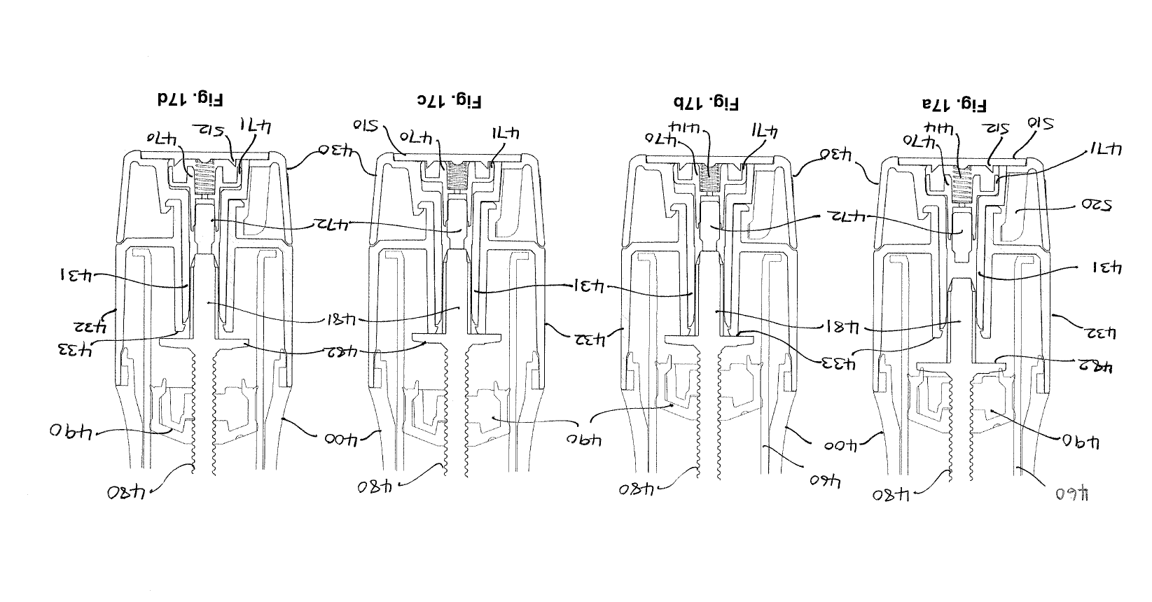

FIGS. 17a to 17d show partial cross-sections through the applicator of FIG. 16a from insertion of the cartridge to priming and back-winding;

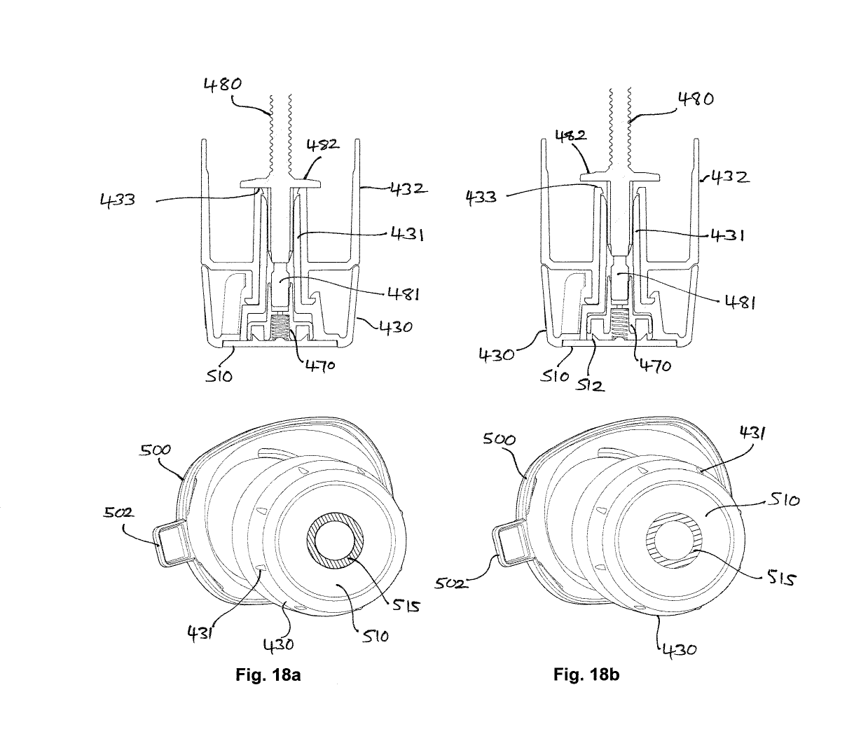

FIG. 18a is a view from below of the applicator primed and ready for use as in FIGS. 17b and 17c;

FIG. 18b is a view from below of the applicator in storage mode as in FIG. 17d;

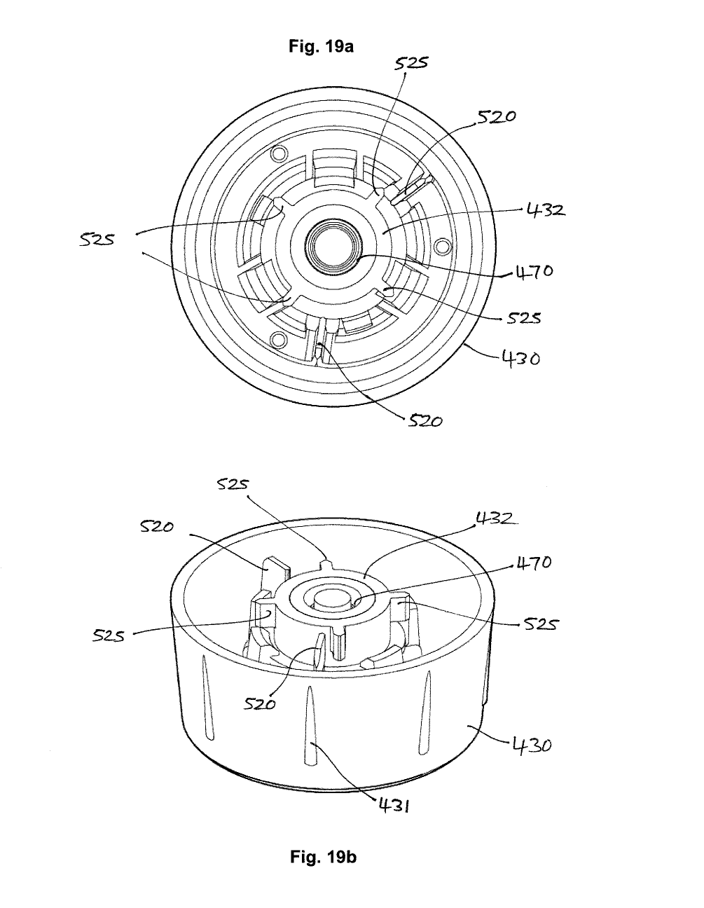

FIG. 19a is a plan view of part of the dial assembly of the applicator of FIG. 16a showing a clicker mechanism;

FIG. 19b is a perspective view from above of the partial dial assembly and clicker mechanism of FIG. 19a; and

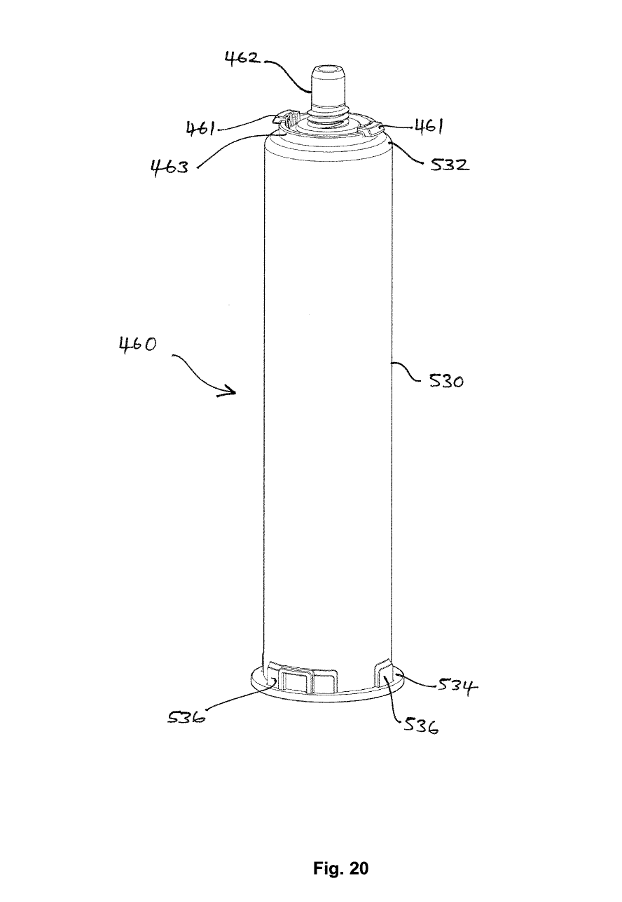

FIG. 20 is a perspective view of a cartridge for use in an applicator according to the invention.

Turning first to FIG. 1a, there is shown an applicator 1 having an applicator head 5 with a smooth, convex, metal applicator face 51 with a central aperture 153. The aperture 153 is sealed by an elastomeric slit valve 71 on the cartridge (not shown). The applicator face 51 is enlarged as compared to the diameter of the applicator head 5 where cartridge is inserted. The shoulder portion 55 behind the applicator face 51 provides a rest surface against which a user exerts fingertip pressure when the applicator is being used and also shields the user's fingers from contact with medicament in the replaceable cartridge as it is dispensed through the slit valve 71 on to the applicator face 51.

The cartridge is enclosed in a housing 40 that fits with the applicator head 5. A dial knob 30 is attached to the housing 40 and is moveable from a decoupled position shown in FIG. 1a in which it can freely rotate without actuating the drive member (not shown) inside the cartridge.

FIG. 1b shows the applicator of FIG. 1a with the dial knob 30 in a coupled position. Rotation of the knob 30 actuates threaded drive member (not shown) inside the cartridge to advance a piston (not shown) and dispense medicament through the valve 71 onto the applicator face 51 from where it may be spread over the user's skin as desired. A single rotation of the dial knob 30 dispenses a predetermined dose of medicament, typically 0.5 g or 1.0 g doses, depending on the pitch of the threaded drive member.

FIG. 1c shows a replaceable cartridge 60 for use with the applicator 1 of FIGS. 1a and 1b The cartridge 60 has an integral valve 71 that seals the aperture 153 in the applicator face 51 when the cartridge 60 is inserted in the applicator head 5 via the cartridge port (not shown). When the medicament in the cartridge 60 is fully dispensed, the cartridge 60 is disposed of and a new one complete with new valve 71 fitted to the applicator head 5.

In FIG. 2a, there is shown in cross-section an applicator 1 that includes a housing 40 which can co-operate with the applicator head 5 to retain the cartridge 60 in its fully inserted position. The applicator head 5 and housing 40 are provided with complementary bayonet fixings in the form of opposing L-shaped slots on the tubular portion of the applicator head 5 and opposing pins on the housing 40, once a cartridge 60 is inserted the housing 40 is then slid on to the applicator head 5 and then rotated to secure the head 5 and housing 40 together.

The applicator head 5 has a cartridge port 53 having a tubular portion extending from behind the applicator face 51 having dimensions for accommodating the neck 62 of a cartridge 60. The cartridge port 53 includes inclined shoulder portion 59 against which a corresponding shoulder portion 69 at the discharging end of the cartridge 60 nests when fully inserted. The neck 69 of the cartridge 60 has an external screw thread that screws onto a complementary internal screw thread on the tubular portion.

On the uppermost surface of the neck 69, that is the leading edge of the cartridge 60, there is a face seal 68 which seals against inwardly directed flange 61 on the cartridge port 53 when the cartridge 60 has been screwed on to the applicator head 5.

Inside the cartridge 60 is a threaded drive rod 80 that terminates in a base portion 81 having an polygonal aperture 82. Around the drive rod 80 is piston 90 having an internal threaded bore 91 that interengages with the threaded drive rod 80 and provides a seal therewith. O-ring seals 82 are provided around the outer circumference of the piston 90 to seal against the inner cartridge walls.

The housing 40 includes a rotatable dial knob 30 at the end thereof that has a polygonal spindle 31 projecting into the housing 40 for co-operating with the polygonal aperture 82 in the base of the drive rod 80 when the cartridge 60 is inserted and the housing 40 fitted to applicator head 5. A re-settable counter mechanism is incorporated in the housing 40.

The applicator of FIG. 2b is similar to that of FIG. 2a, but instead of the cartridge 60 fitting into the applicator head 5 by screwing in, the cartridge 60 is simply slid into the cartridge port 53 and is a resilient fit therewith. The neck of the cartridge 60 has an O-ring seal 69 that seals against the tubular portion of the cartridge port 53. Instead of a bayonet fitting, the housing 40 screws on to the applicator head 5 by virtue of complementary screw threads on each part.

In FIG. 2c, there is no separate housing as such. Instead, the cartridge 60 screws directly on to the applicator head 5 in the same way as it does in FIG. 2a. The dial knob 30 is carried on the cartridge 60.

The applicator of FIG. 2d includes an integral housing 400 that extends from the applicator head 5 to accommodate a cartridge 60 which is fitting by pushing in to the tubular portion of the cartridge port 53 in a similar manner to FIG. 2b. The dial knob 30 has release buttons that engage in the housing walls when the cartridge 60 is fully inserted to retain the cartridge 60 in position against the applicator head 5. Depressing the release buttons after the cartridge 60 has been discharged allows the cartridge 60 to be released for disposal and the applicator to be re-used.

FIGS. 3a and 3b illustrate how the same applicator can be used to accommodate different sized replaceable cartridges 60a, 60b. The cartridges 60a, 60b have the same length but different diameters, hence contain different volumes of medicament when charged. The discharging end of the cartridges 60a, 60b are provided with a universal fitting 64 for connection with the same applicator head 5. Additionally, the base portion 81 of the drive rod 80 in each cartridge 60a, 60b is provided with an aperture 82 of the same size so that the same dial knob 30 on the applicator housing 40 is able to rotate the threaded drive rod 80 in both cartridges 60a, 60b.

The pitch of the thread is the same in both sized cartridges 60a, 60b hence a single rotation of the dial knob 30 will result in different doses of medicament being dispensed. Thus for 30 g and 60 g cartridges of the same length, one revolution will result in twice as much medicament being dispensed from the 60 g cartridge 60b as compared to the 30 g cartridge 60a. This is useful when a patient requires, say only a 0.5 g dose, as opposed to a 1.0 g dose. Of course, providing the threaded drive rods 80 with different pitches makes it possible for the same dose to be delivered.

A range of re-usable applicator heads 5a, 5b and a range of disposable cartridges 60a, 60b, 60c, 60d of varying volumes are shown in FIG. 4. Where the area of skin to be treated is relatively small, an applicator head 5b having a relatively small applicator face is available and a smaller capacity cartridge 60d is attached. On the other hand, an applicator head 5a having a larger diameter applicator face is suitable for use with a range of larger volume cartridges 60a, 60b, 60c each of which have a universal fitting 64 at the neck portion. Each of larger volume cartridges 60a, 60b, 60c, 60e include an integral counter mechanism 110 or a transparent window 120 through which the level of medicament remaining in the cartridge can be observed. The window 120 is UV tinted to prevent degradation of the medicament in the cartridge 60e.

Various counter options for indicating how much medicament has been dispensed from the applicator or how much medicament remains are illustrated schematically in FIGS. 5a to 5g. FIG. 5a includes a simple mechanism that emits an audible click, or multiple clicks, as each revolution or fraction of revolution of the dial is completed, but does not include a counter mechanism as such. Audible mechanisms are known and are readily understood by a skilled person.

FIG. 5b shows an applicator having a window 130 through which a planar, radial scale 132 is displayed. As each dose of medicament is dispensed, the decreasing scale 132 advances incrementally to indicate the transition between full and empty.

In FIG. 5c the applicator again has a window 130 through which a dosing scale 134 is displayed. Specifically, the counter scale 134 indicates the total volume of medicament that has been dispensed. Such a counter mechanism enables a user to monitor a particular dose if, for example, the required dose requires several rotations of the dial knob, or merely a fraction of a single rotation.

FIG. 5d differs from the counter mechanism shown in FIG. 5c in that the total volume of medicament remaining in the cartridge is indicated on the counter scale 136. This is helpful to indicate to a user the number of doses remaining before the cartridge is empty.

FIG. 5e shows an applicator having an automatically re-setting dose counter 138. The dial knob 30 on the applicator is pulled out and twisted to dispense the medicament and thus prevents accidental actuation. The dose counter is re-set to zero after each actuation.

FIG. 5f shows an applicator having a digital counter display 139 in a housing window 130. The digital display 139 includes information on the date and dose dispensed that day; the display 139 can be switched to additional modes so that a user can review the dosing history and/or the volume of medicament dispensed and remaining.

The counter indicator 140 on the applicator of FIG. 5g is located on the base of the dial knob 30 and provides an indication of the fill level in the cartridge. When the applicator is stored it is rested in a stand with the applicator head lowermost. Thus a user can easily view the indicator 140 when the applicator is stood and initially picked up from the stand.

The applicators illustrated in FIGS. 6a to 6f by cross-sectional and bottom end views are respectively provided with various different fill indicators. In FIG. 6a, the fill indicator simply comprises several apertures 145 through the base of the dial knob 30 through which a user can view the advance of the piston 90 up the cartridge 60 as the medicament is dispensed.

The fill indicator of FIG. 6b involves an inner tube 150 inside a housing for the cartridge 60 around which an outer ring 152 is driven as the dial knob 30 is rotated. An elongate window in the housing wall allows a user to observe the position of the ring 152 which is driven up as the medicament in the cartridge 60 is dispensed.

In FIG. 6c, the base of the dial knob 30 has an aperture 170 and the hollow dial knob houses a counter mechanism including drive wheel 160 and a disc 162 carrying dosing indicia which is advanced by the drive wheel 160 as the dial knob 30 is rotated. A user is able to view the volume of medicament that has been discharged by the specific indicia displayed through the window 170.

The fill mechanism of FIG. 6d comprises a ribbon 180 having a first end attached to the trailing face of the piston 90, the ribbon 180 extending around the base of the cartridge 60 and against the external cartridge wall. The second end of the ribbon is not fixed. As medicament is dispensed and the piston 90 advances up the cartridge 60, the piston 90 drags the ribbon behind it. A window 182 in the housing 40 permits a user to see the ribbon 180, so that as the second end of the ribbon descends down the external cartridge wall as the medicament is dispensed, a user can see how much the cartridge 60 has been depleted by the position of the second end.

FIG. 6e shows an applicator having a Geneva wheel counter indicator 190 on the dial knob 30. The dial knob 30 acts as a drive wheel and carries a pin that reaches into a slot of the driven wheel and advances it by a single step with each revolution of the dial knob 30. The driven wheel carries dosing indicia on its side wall around its circumference viewable through the aperture in the wall of the housing.

In FIG. 6f, the fill indicator is a harmonic drive mechanism 195 in which the dial knob 30 has an elliptical disc fixed internally to its base inside the knob that acts as a wave generator. As the dial knob 30 is rotated, the elliptical disc flexes a spline that has teeth around its outer circumference that mesh with teeth on the inner circumference of a counter wheel. For every full rotation of the elliptical disc (equivalent to one revolution of the dial knob), the flexible spline rotates in the opposite direction but only by an incremental amount and the outer ring is similarly advanced. Dosing indicia displayed around the circumference of the outer wheel is viewable through the aperture in the wall of the housing.

FIGS. 7a to 7c illustrate a further form of fill indicator in the form of a scratch indicator. In this arrangement, the piston 90 carries a blade 200 extending laterally from the trailing edge of the piston 90 that scratches the internal wall of the cartridge 60 as the piston 90 rises. The internal wall of the cartridge 60 is coated with a material that is scraped off under the action of the blade 200. The cartridge 60 carries a graduated scale, adjacent the blade member, so a user can determine the volume of medicament used or remaining by observing the advance of the score mark 202 on the cartridge wall.

FIG. 8 illustrates an advanced piston construction in which the lead surface of the piston 90 is inclined to match an inclined shoulder portion at the discharging end of the cartridge so that the piston 90 is able to advance all the way up the cartridge 60 and discharge all the medicament without having to waste any.

The piston 90 includes leading drive nut 901 rotatably engagable with the threaded drive rod 80, drive seal 903 abutting the drive nut 901 seals around the thread of the drive rod 80 behind the drive nut 901, and resilient plug 905 surrounding the drive nut 901 exerts a sealing force against the internal cartridge walls. Two O-ring seals 82 around the outer circumference of the piston 90 seal against the internal tube walls of the cartridge 60.

The cartridge tube has a counterbore 907 adjacent its second end to permit air to escape when the piston 90 is inserted and to allow excess medicament to squirt out in the event if overfilled.

FIGS. 9a to 9f show a flexible cartridge 600 and an applicator therefor. A flexible, tubular bag 601 made of foil is bonded to a substantially rigid cap 603 at its discharging end. The end cap 603 carries an external screw thread 605 for screwing on to an internal screw thread in the cartridge port of applicator head.

The applicator has a plate 290 having two pegs 291a, 291b extending on the either side. The pegs 291a, 291b of the plate 290 pass through vertical slots in a first inner sleeve 293 which houses the cartridge 600, extending into spiral grooves on a second, outer sleeve 295. The outer sleeve 295 is attached to the dial knob 30 and hence rotates with the dial knob 30 to drive the plate 290 longitudinally up the applicator compressing the bag 601 to dispense medicament through the valve 71 onto the applicator face 51. FIG. 9d shows the initial position of the plate 290 when the bag 601 is full, FIG. 9e the position when partially discharged and FIG. 9f the position when fully discharged.

FIGS. 10a to 10g show a different form of flexible bag cartridge 660 and applicator therefore. In this arrangement, as seen from FIGS. 10a to 10c, the flexible bag 661 has the same substantially rigid cap 603 at its discharging end, but this time includes an elongate hollow channel 665, extending from the second end to accommodate a threaded drive rod 80 of the applicator.

When the bag 661 is inserted in the applicator, the drive rod 80 extends along the channel 665. In use, the disc 670 around the threaded drive rod 80 is driven up towards the discharging end as the dial knob 30 is rotated and compresses the bag 661 to dispense medicament. FIG. 10e shows the initial position of the disc 670 when the bag is full, FIG. 10f the position when partially discharged and FIG. 10g the position when fully discharged.

FIGS. 11a to 11e illustrate an applicator including a dial knob deactivation mechanism that requires a “push, engage and twist” action to dispense medicament. The mechanism allows the medicament in the cartridge 60 to expand or contract as it is subjected to different ambient temperatures.

In FIG. 11a, there is shown a filled cartridge 60 at a typical temperature of 25° C. The position of the piston 90 within the cartridge 60 can be observed at Level 0 corresponding to the position of the O-ring seal 82 of the piston. The cartridge wall extends beyond the lower face of the drive rod base 81 to allow for any thermal expansion of the medicament that takes place either when the cartridge 60 is in storage or once inserted in an applicator.

FIG. 11b shows the cartridge 60 inserted in an applicator which is in a deactivated state, with the spindle 31 on the dial knob 30 decoupled or disengaged with respect to the aperture 82 on the drive rod base 81. Due to the fact that the dial knob 30 is decoupled, allowing it to spin freely without driving the piston 90, the medicament is able to undergo thermal expansion. At an ambient temperature of 40° C., the O-ring 82 of the piston has descended to Level 1 (lower than Level 0).

As shown in FIG. 11c, the dial knob 30 can be moved into engagement with the drive rod base 81 by pushing in and then activated by twisting (rotating) to dispense medicament. After dispensing, the dial knob 30 is pulled out to return it to the deactivated state.

Equally, if the ambient temperature drops to 0° C., the medicament in the cartridge 60 can undergo shrinkage as indicated by Level 2 (higher than Level 0) in FIGS. 11d and 11e, which illustrate respectively decoupled and coupled states of the applicator.

FIGS. 12a and 12b illustrate an arrangement where the applicator head 5 is moveable on a housing 40 to allow medicament to be dispensed on to the applicator face 51. The applicator head 5 is a moveable unit, so that on activation of the drive mechanism (not shown), the head 5 is moved from a first, rest position in which a pin 171 on the cartridge 60 seals the aperture 153 in the applicator face 51 (FIG. 12a) to the second position raised above the pin 171 (FIG. 12b). Movement from the first to the second positions is caused by the pressure exerted on the rear surface of the applicator face 51 by the medicament as the piston is driven up the cartridge.

In this arrangement, the cartridge 60 is a push-fit in the tubular portion of cartridge port 53 to allow relative movement between the port 53 on the applicator head 5 and the cartridge 60. The tubular portion of the cartridge port 53 has a length sufficient to accommodate and retain the cartridge neck 62 when the head 5 is in both the rest and raised positions.

The applicator head 5 is effectively a floating head on the housing 40 and the complementary engagement mechanism between the head 5 and housing 40 permits limited movement of the applicator head 5 away from the housing 40.

FIGS. 13a and 13b show an alternative arrangement in which the pin 271 that seals the aperture 153 on the applicator face 51 is mounted on the applicator head 5 adjacent the cartridge port 53, but independent of the applicator face 51. The applicator face 51 is moveable on the applicator head 5, from a first, rest position in which the pin 271 seals the aperture 153 in the applicator face 51 (FIG. 13a) to a second, raised position above the pin 271 (FIG. 13b). In this arrangement, instead of the entire applicator head 5 rising under pressure from the medicament when the dial knob (not shown) is rotated, the force behind the applicator face 51 just raises the applicator face 51 itself. In the raised position, a fluid pathway opens up around and above the pin 271 to allow medicament to be dispensed on to the applicator face 51.

In this latter arrangement, the cartridge 60 remains stationary in the cartridge port 53 once inserted, and the applicator face 51 acts as a floating fascia on the applicator head 5.

The pin 271 on the applicator head 5 terminates in a sharp tip 273 to puncture a frangible seal (not shown) on the cartridge 60 when the sealed cartridge is fitted in the applicator.

FIGS. 14a to 14c illustrate alternative means for accommodating thermal expansion of the medicament which may be incorporated instead of the decoupling solution shown in FIGS. 11a to 11e, or in addition thereto.

In particular, FIG. 14a shows an applicator with removable cover 300 which, when fitted over the applicator head 5, creates a space 305 between the underside of the cover 300 and the applicator face 51 in the region of the aperture 153. In the event that the medicament undergoes thermal expansion within the cartridge 60, medicament is able to leak out through the valve 71 and be retained in the space. A user then simply wipes clean the applicator face 51 prior to dispensing a dose of medicament.

The applicator of FIG. 14b is provided with an escape passage 310 in the wall of the cartridge port 53 and through which medicament can seep if subject to thermal expansion.

The applicator of FIG. 14c includes an expansion chamber 320 in the piston 90 which chamber is in fluid communication with the medicament in the cartridge. The expansion chamber 320 has sufficient capacity to accommodate the maximum possible expansion that the medicament may be subject to when the fully charged.

FIGS. 15a to 15c illustrate a mechanism for preventing counter-rotation of the drive rod 80 in an applicator that includes a decoupling mechanism. In FIG. 15a, the dial knob 30 is freely rotatable since its spindle 31 is not in co-operation with the aperture 82 in the base 81 of the drive rod 80. In this position, the applicator is effectively prevented from accidentally dispensing medicament.

The base portion 81 of the drive rod includes a collar 880 on which the radially extending ribs are carried. The collar 880 is provided with one or more longitudinal slits to permit splaying when a force is exerted on the collar 880 from within.

The dial knob 30 includes an inner ring 380 concentric with, but spaced from, the outer dial knob wall and which serves to splay the collar 880 of the drive member 80 when the dial knob 30 is brought into coupling relationship with the drive rod 80 as shown in FIG. 15b. The external side wall of the inner ring 380 is tapered to facilitate insertion of the ring 380 in the collar 880 and to gradually splay the collar 880 as the taper is advanced within.

As will be better seen from FIG. 15c, ribs are also provided on the internal cartridge wall with the result that as the tapered portion of the dial knob side wall is advanced inside the collar, the ribs on the collar are forced out and between the ribs on the internal cartridge wall. This overlapping of the ribs allows the drive rod to be rotated under the action of the dial knob and drive the piston forwards and prevent “re-winding” along the drive rod.

FIGS. 16a to 16c illustrate an applicator 401 according to the invention incorporating both a valve switch 450 and back-wind indicator mechanism.

As seen in FIG. 16a, the applicator 401 has an applicator head 405 with integral applicator body 400 and a dial assembly 440 in the form of a dial knob 430 rotatably mounted on a dial body 432. The dial knob 430 has spaced apart raised formations 431 around the outer surface to assist gripping. The applicator head 405 has recesses 452 for securing a projection on a tab 502 of a head cover 500 used when the device is stored, as shown in FIG. 16c. The applicator face 451 includes a resilient insert 456 having an aperture 471 closed by a pin valve 472. The applicator 401 has a slider switch 510 for operating the valve 472 to open and close the aperture 471. The slider switch 510 has a projection 511 to facilitate fingertip use.

FIG. 16b is a cross-section through the applicator of FIG. 16a fitted with a cartridge 460 and primed and ready for use. The cartridge has a neck portion 462 that extends through the cartridge port 453 so that the open, discharging end is aligned with the aperture beneath the pin of the pin valve 472. The cartridge 460, shown more clearly in FIG. 20, has opposing lugs 461 projecting laterally from a raised ring 463 on the cartridge shoulder which are captured by slots in the applicator head 405 adjacent the cartridge port 453 when the cartridge is inserted and rotated in the manner of a bayonet mount. The cartridge 460 is fitted by inserting it in the open end of the applicator body 400 and pushing it in so that the shoulder portion of the cartridge abuts an opposing surface at the cartridge port 453, at which point the cartridge is twisted (rotated) relative to the applicator body so that the lugs 461 are captured and the cartridge is prevented from moving away from the cartridge port. The process is reversed for removing the cartridge, such as when a depleted cartridge is to be replaced with a fresh one.

The cartridge 460 has a threaded drive rod 480 and a piston 490 sealingly mounted on the drive rod and in sealing engagement with the cartridge wall. The drive rod 480 terminates in an end portion 481 which projects beyond the piston 490 and is captured in the hollow spindle 431 of the dial knob 430 of a dial assembly 440. The dial knob 430 is rotatably mounted on a dial body 432 which is removably connected to the applicator body 400 to allow insertion and removal of the cartridge 460. The end portion 481 of the drive rod 480 is splined and is rotationally fixed within the spindle 431 which has complementary features that engage with the splined portion. A flange 482 on the drive rod 480 between the end portion 481 and the threaded portion inside the cartridge rests against a stop surface 433 of the dial body 432 so that forward rotation of the dial knob 430 drives the piston 490 up the cartridge to dispense medicament.

In FIG. 16c, the dial knob 430 has been backwound resulting in the drive rod 480 being raised in the cartridge so that the flange 482 is no longer against the stop surface 433 of the dial body 432. This is the normal storage configuration for the applicator and hence any thermal expansion of the medicament in the cartridge can be accommodated due to the drive rod 480 having the freedom to move back down the cartridge body.

The slider switch 510 has an arm 512 that extends through a slot in the applicator head 405 and cooperates with a valve riser member on which pin valve 472 is supported. In the position shown in FIG. 16b, the pin valve 472 is retracted so that the aperture 471 in the applicator face 451 is open to allow medicament to be dispensed.

The drive rod 480 of the cartridge 460 is backed down so that the stop flange 482 abuts the stop flange 433 on the dial body 432 around the dial knob spindle 431, so any forwards rotation of the dial knob 430 will result in medicament being discharged from the cartridge 460 and dispensed through the aperture 471 in the applicator face 451 because the piston 490 in the cartridge will move in the dispensing direction.

In FIG. 16c, the aperture 471 is shown closed by virtue of the slider switch 510 being reversed so that the pin valve 472 is raised on the valve riser member. In addition, the drive rod 480 is raised above the stop flange 433 of the dial body 432 following back-winding of the dial knob 430. Thus the applicator is shown in the storage position. A removable head cover 500 is fitted to the applicator head 405, the projections on the underside of the head cover being engaged in the recesses 452 of the head to hold the cover in place.

At the opposite end of the applicator, the dial assembly 440 accommodates a backwind indicator mechanism. The mechanism includes an indicator member 470 having an elongate portion 472 extending into the bore of the dial knob spindle 431 which is in contact with the end face of the end portion 481 of the drive rod 480. The indicator 470 has a coloured ring portion 471 concentric with a lens ring 512 on the internal face of the translucent end face 510 of the dial knob 430. A spring 414 is retained between the indicator member 470 and the dial knob end face 510, the spring being compressed by the force exerted by the end portion 481 of the drive rod when the applicator is primed for use (as in FIG. 16b) allowing the indicator ring to rest against the dial knob end face 510 so that its colour is reflected on to the external surface through a clear central lens ring 515. In the backwound position of FIG. 16c, the end portion of the drive rod 481 is raised with respect to the dial knob 430 and the spring 414 expands lifting the indicator member 470 and hence the indicator spring off the internal end face 510 of the dial knob 430. The colour of the dial knob 430, which contrasts with the colour of the indicator ring 470, is now reflected through the central lens ring 512 onto the external face 510 of the dial knob. A user of the applicator is therefore able to tell whether the applicator is primed and ready for use or is in the storage (backwound) mode according to the colour reflected onto the external face.

A sequence showing the various positions of the drive rod 480 and backwind mechanism is displayed in FIGS. 17a to 17d. FIG. 17a illustrates the position of the drive rod 480 in the cartridge 460 following initial insertion of the cartridge into the applicator. The flange 482 on the drive rod 480 abuts the underside of the piston 490 effectively creating a seal with the piston thereby helping keep the medicament within the cartridge out of contact with the air. The projecting end 481 of the drive rod, whilst captured and rotationally fixed with respect to the spindle 431 of the dial knob, is out of contact with the indicator member 470 of the backwind mechanism. Thus the indicator member 470 is in a raised position under the action of the indicator spring 414. The colour of the dial knob 430 is reflected onto the external end face 510 of the dial knob 430 indicating that the applicator is not in a dispensing mode.

Following insertion of the cartridge, the dial knob 430 is rotated to draw the drive rod 480 down towards the dial knob and in so doing presses against the dowel 472 that extends from the indicator member. The ring 471 of the indicator member is hence pressed against the internal end face 510 of the dial knob 430 whereupon the colour of the indicator ring which is different to that of the dial knob is reflected onto the external end face, indicating that the applicator is primed ready for dispensing as shown in FIG. 17b.

When the flange 482 on the drive rod 480 abuts the stop surface 433 of the dial body 432 the drive rod is prevented from further downward movement, thus any further rotation of the dial knob causes the piston 490 to rise as shown in FIG. 17c and results in medicament being dispensed onto the applicator face 451. During dispensing, the end face 510 of the dial knob 430 still has the colour of the indicator ring 471 reflected onto it since the indicator spring 414 is kept under compression.

FIG. 18a is a view from the underside of the applicator (with cap 500) displaying a ring of reflected colour of the indicator ring 471 on the end face 510 as would appear when the applicator is primed and in use as shown in FIGS. 17b and 17c.

After a dose of medicament has been dispensed, the applicator may be returned to its storage mode by backwinding the dial knob 430. Backwinding results in the drive rod 480 rising up so that the flange 482 of the drive rod is lifted away from the stop surface 433 with the piston 490 remaining stationary, thereby allowing a degree of thermal expansion of the medicament. As will be seen from FIG. 17d, the indicator ring 471 is lifted off the end face 510 of the dial knob 430 by the spring 414 so that the colour of the dial knob is again reflected onto the external face 510 indicating that the applicator has been returned to a storage mode.

FIG. 18b is a view of the underside of the applicator (with cap 500) displaying a ring of reflected colour of the dial knob 430 on the end face 510 as would appear when the dial knob is backwound for storage.

FIGS. 19a and 19b are partial plan and perspective views respectively of the dial assembly 440 of the applicator of FIGS. 16a to 16c to show more clearly the clicker mechanism housed within the dial knob 430. The dial knob 430 carries two clicker arms 520 and the dial body 432 has four equally spaced clicker posts 525. As the dial knob is rotated, the clicker arms 520 ride over the posts 525 which, being on the dial body 432, remain stationary. In the arrangement shown, each complete revolution of the dial knob 430 produces eight audible clicks. Accordingly, a user will be able to determine the dose delivered according to the number of clicks.

FIG. 20 is a perspective view of a cartridge 460 similar to that shown inserted in an applicator as in FIGS. 16a to 16c. The cartridge 460 is comprised of an elongate tube 530 having a shoulder portion 532 on which a pair of opposing lugs 461 are carried for engaging with complementary slots on an applicator. The cartridge nozzle 462 at the dispensing end is threaded for securing a cartridge cap (not shown) to maintain the medicament within the cartridge sealed from the atmosphere. The base of the cartridge has an outwardly directed supporting flange 534 for enabling the cartridge to be stood on end in storage. The inside of the non-dispensing end of the cartridge tube is provided with air vents (not shown) and corresponding projections 536 on the external wall are provided to maintain a substantially constant wall thickness around the air vent features.

While a number of examples have been chosen to illustrate the present invention, it will be apparent to those skilled in the art that various changes and modifications can be made herein without departing from the spirit and scope of the invention. For example, while the dial knob is preferably manually rotatable, it will be appreciated that it could alternatively be driven to rotate by means of an electronic motor. Such a motor may be housed within the dial assembly. Further, while the drive member is typically a threaded member, such as a threaded drive rod, other forms of drive member are contemplated, including for example a torque-spring drive member Moreover, the applicator may be used to apply medicaments for any number of skin complaints, including for example dermatitis and eczema, as well as psoriasis. Combinations of features described hereinbefore, whether or not exemplified, are also regarded as falling within the scope of the invention. Thus, the foregoing description of the embodiments according to the present invention are provided for illustration only, and not for the purpose of limiting the invention.

1. An applicator for topically applying a semi-solid medicament, the applicator comprising:
an applicator head having an applicator face for spreading medicament across the skin;
a sealable aperture on the applicator face; and
a drive mechanism for forcing medicament out through the aperture, wherein:
the applicator head includes a cartridge port at the rear of the applicator face for receiving a dispensing end of a cartridge charged with medicament; and
the drive mechanism comprises a dial knob rotatable around a longitudinal axis of the applicator for advancing a piston along a drive member towards the applicator head to force medicament from the cartridge and thereby dispense a predetermined dose of medicament onto the applicator face.

2. The applicator of claim 1, wherein the applicator face has a diameter greater than the diameter of the applicator head in the region of the cartridge port thereby to create a shoulder portion behind the applicator face for shielding a user's fingers from medicament on the applicator face and medicament applied to the skin when the applicator is in use.

3. The applicator of claim 1, wherein the applicator face is inclined with respect to the longitudinal axis of the applicator.

4. The applicator of claim 1, further comprising a dial assembly including the dial knob and a dial body on which the dial knob is rotatably mounted.

5. The applicator of claim 4, further comprising an applicator body extending from the applicator head and co-operable with the dial body to enclose the cartridge, and wherein the dial body is releasably attachable to the applicator body for allowing insertion and removal of the cartridge.

6. The applicator of claim 5, wherein the applicator body is adapted to accommodate a portion of the cartridge so that a non-dispensing end of the cartridge extends beyond the end of the applicator body and into the dial body when fitted to the cartridge port.

7. The applicator of claim 1, wherein the dial knob is adapted to capture an end portion of the drive member such as to permit reciprocal movement of the drive member and to rotate the drive member with the dial knob.

8. The applicator of claim 1, wherein the dial knob comprises an outer dial ring and an inner spindle for linear coupling with an end portion of the drive member, the inner spindle having a profile complementary to that of the drive member such that the drive member is rotated with the spindle upon operation of the dial knob

9. The applicator of claim 8, wherein the inner spindle has a bore for receiving the end portion of an axially aligned drive rod, the bore having a non-circular cross-section.

10. The applicator of claim 1, wherein the applicator includes an abutment for limiting the extent of longitudinal movement of the drive member in a reverse direction.

11. (canceled)

12. The applicator of claim 1, wherein the dial knob is rotatable about a longitudinal axis of the applicator in a forwards direction for priming the applicator and bringing the applicator into a dispensing mode and rotatable in the reverse direction (back-winding) for returning the applicator to a non-dispensing, or storage mode.

13. The applicator of claim 12, wherein the dial knob is rotatable in a reverse direction by one revolution or more for accommodating any thermal expansion of medicament that might occur during storage.

14. The applicator of claim 1, further comprising a backwind status indicator mechanism for indicating whether the applicator is in a dispensing mode or a storage mode.

15. The applicator of claim 14, wherein the indicator mechanism is operable by means of the drive member, such that a first storage status is indicated when the drive member is advanced by back-winding of the dial knob and a second, dispensing status is indicated when the drive member is reversed by winding the dial knob forwards.

16. The applicator of claim 14, wherein the indicator mechanism is housed within a dial assembly.

17. The applicator of claim 14, wherein the indicator mechanism comprises an indicator member that is pressed against the end face of the dial knob by the drive member when the applicator is primed and ready for use and is raised above the end face when the applicator is in the storage mode.

18. The applicator of claim 17, wherein the indicator mechanism further comprises an indicator spring interposed between the indicator member and the end face of the dial knob, whereby when the dial knob is forwards rotated the drive member presses the indicator member against the end face, and when the dial knob is counter-rotated or back-wound, the spring urges the indicator member away from the end face.

19. The applicator of claim 17, wherein the end face of the dial knob is translucent or transparent for observing the position of the indicator member.

20. The applicator of claim 19, wherein the end face of the dial knob comprises an end cap having a clear central lens ring on an inner surface thereof which lens ring reflects the colour of the surface proximate the lens ring onto the external face of the end cap, and wherein the indicator member comprises a coloured ring concentric with the lens ring that is pressed against the end face under the action of the drive rod when the applicator is primed, the colour of the indicator ring being reflected onto the external face of the end cap.

21. The applicator of claim 1, wherein the aperture is sealable by means of a valve.

22. The applicator of claim 21, further comprising a valve switch for switching the valve between sealing and open positions.

23. The applicator of claim 22, wherein the valve comprises a pin valve and the applicator further comprises a valve riser member operable by the valve switch to raise the valve to close the aperture and lower the valve to open the aperture.

24. The applicator of claim 1, further comprising a dosing indicator for indicating when a dose has been dispensed.

25. The applicator of claim 24, wherein the dosing indicator comprises a clicker mechanism for producing a fixed number of audible clicks per complete revolution of the dial knob, and wherein the clicker mechanism comprises one or more clicker arms on the dial knob of a dial assembly, and a plurality of equally spaced clicker posts on a dial body of the dial assembly.

26. (canceled)

27. The applicator of claim 1, further comprising a retaining mechanism for securing the cartridge to the applicator head in a fully inserted position.

28. The applicator of claim 1, wherein the cartridge port includes a coding feature for coding with a complementary coding feature on the cartridge such that only cartridges carrying the requisite coding feature may be fitted to the cartridge port.

29. The applicator of claim 1, further comprising a cartridge charged with medicament.

30-49. (canceled)

50. An applicator kit for use in treatment of a skin condition, the applicator kit comprising an applicator according to claim 1 in combination with a Patient Information Leaflet comprising operating instructions for the applicator.

51. The applicator kit of claim 50, further comprising a stand having a hollowed recess for receiving the applicator head of the applicator, whether or not fitted with a cartridge, for storage purposes.

52. The applicator kit of claim 50, further comprising one or more replaceable cartridges charged with medicament for fitting to the applicator head via the cartridge port.

53-55. (canceled)

56. A method of applying a dose of a semi-solid medicament to a skin area to be treated with an applicator assembly comprising an applicator and a medicament cartridge, wherein:
the applicator comprises:
an applicator head having an applicator face for spreading medicament across the skin, the applicator head having a cartridge port at the rear of the applicator face for receiving a dispensing end of a medicament cartridge charged with the semi-solid medicament;
a sealable aperture on the applicator face; and
a drive mechanism for forcing medicament out through the aperture, the drive mechanism having a dial knob rotatable around a longitudinal axis of the applicator for advancing a piston along a drive member towards the applicator head to force medicament from the cartridge and thereby dispense a predetermined dose of medicament onto the applicator face;
the medicament cartridge comprises:
an elongate container having a discharging end that cooperates with the cartridge port of the applicator and a second end opposite the first end;
a drive rod extending longitudinally in the container; and
a piston mounted on the drive rod and in sealing contact with an inner wall of the container for advancing along the container to dispense medicament from the discharging end upon rotation of the drive rod in a forward direction by the dial knob of the applicator; and
the drive rod of the medicament cartridge includes an end portion that projects beyond the piston, the end portion being adapted for coupling with the dial knob of the applicator such that the drive rod is rotationally fixed relative to the dial knob and such that the drive rod is rotated with the dial knob and is permitted relative axial movement relative to the dial knob shaft to raise or lower the drive rod with respect to the dial knob during priming and back-winding operations of the applicator assembly,
the method comprising:
(i) fitting the dispensing end of the medicament cartridge to the cartridge port of the applicator;
(ii) coupling the drive rod of the medicament cartridge with the dial knob of the applicator;
(iii) rotating the dial knob in the forward direction to prime the applicator;
(iv) further rotating the dial knob in the forward direction to dispense a dose of the semi-solid medicament through the aperture and onto the applicator face;
(v) applying the dose of the semi-solid medicament dispensed in (iv) to the skin; and
(vi) using the applicator face to spread the medicament over the skin area to be treated.

57. The method of claim 56, further comprising:
(vii) rotating the dial knob in a reverse direction to raise the drive rod and place the applicator in a storage mode.

58. The method of claim 56, further comprising:
moving a valve switch to open the aperture on the applicator face prior to (iv); and
moving the valve switch to close the aperture prior to (v).
